(19) United States
(12) Patent Application Publication
(10) Pub. No.: US 2026/0232268 A1
(43) Pub. Date: Aug. 13, 2026

(54) PATCH MANUFACTURE

(71) Applicant: WearOptimo Pty Ltd, South Brisbane, Queensland (AU)

(72) Inventors: Anthony Mark BREWER, South Brisbane (AU); Robert Paul SCHARF, Ehningen (DE); Vignesh SURESH, South Brisbane (AU)

(21) Appl. No.: 19/155,216

(22) PCT Filed: Feb. 12, 2024
(86) PCT No.: PCT/AU2024/050089

§ 371 Date: Aug. 8, 2025

(30) Foreign Application Priority Data
Feb. 13, 2023 (AU) 2023900339

(51) Int. Cl. A61B 5/00; A61B 5/145

(52) U.S. Cl. CPC A61B 5/6833; A61B 5/14514; A61B 2562/028; A61B 2562/12

(57) ABSTRACT

A method for manufacturing a patch for application to a biological subject, the method including shaping a formable material to form a plurality of microstructures on a substrate, wherein the microstructures are shaped to breach a functional barrier of the subject.

### BACKGROUND OF THE INVENTION

The present invention relates to a method of manufacturing a patch for application to a biological subject and in one particular example, to a method of manufacturing a microstructure patch for use in performing measurements on, or delivering therapy or stimulation to, a biological subject.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Biological markers, such as proteins, antibodies, cells, small chemicals, hormones and nucleic acids, whose presence in excess or deficiency may indicate a diseased state, have been found in blood serum and their levels are routinely measured for research and for clinical diagnosis. Standard tests include antibody analysis for detecting infections, allergic responses, and blood-borne cancer markers (e.g. prostate specific antigen analysis for detecting prostate cancer). The biological markers may originate from many organ systems in the body but are extracted from a single compartment, the venous blood.

However, this is not suitable for all conditions as often blood does not contain key biological markers for diseases originating in solid tissues, and whilst this problem has been partially overcome by taking tissue biopsies, this is time-consuming, painful, risky, costly and can require highly-skilled personnel such as surgeons.

Another serum-rich fluid is the interstitial fluid (ISF) which fills the intercellular spaces in solid tissues and facilitates the passage of nutrients, biomarkers, and excretory products via the blood stream.

WO2005/072630 describes devices for delivering bioactive materials and other stimuli to living cells, methods of manufacture of the device and various uses of the device, including a number of medical applications. The device comprises a plurality of structures which can penetrate a body surface so as to deliver the bioactive material or stimulus to the required site. The structures are typically solid and the delivery end section of the structure is so dimensioned as to be capable of insertion into targeted cells to deliver the bioactive material or stimulus without appreciable damage to the targeted cells or specific sites therein.

WO2020/069565 describes a system for performing measurements on a biological subject, the system including: at least one substrate including a plurality of plate microstructures configured to breach a stratum corneum of the subject; at least one sensor operatively connected to at least one microstructure, the at least one sensor being configured to measure response signals from the at least one microstructure; and, one or more electronic processing devices configured to: determine measured response signals; and, at least one of: provide an output based on measured response signals; perform an analysis at least in part using the measured response signals; and, store data at least partially indicative of the measured response signals.

Attempts have been made to manufacture microstructures using injection moulding and other similar techniques. Whilst injection moulding can yield low cost high volume structures and microinjection moulding has advance significantly, this technique cannot produce micron or submicron features and does not lend to downstream serial processing such as metallisation or dielectric coating. Whilst semiconductor process can be done on wafer scale or large area substrates, and this offers cost efficiencies and allows for precise downstream processing, either batch processing or potentially inline processing, this process is typically prohibitively expensive.

### SUMMARY OF THE PRESENT INVENTION

In one broad form, the present invention seeks to provide a method for manufacturing a patch for application to a biological subject, the method including shaping a formable material to form a plurality of microstructures on a substrate, wherein the microstructures are shaped to breach a functional barrier of the subject.

In one embodiment the method includes: providing a substrate; depositing a formable material layer on the substrate; forming a plurality of microstructures in the formable material layer, wherein the microstructures are shaped to breach a functional barrier of the subject; and, solidifying the formable material.

In one embodiment the method includes forming the plurality of microstructures by one of: hot embossing; and, imprinting.

In one embodiment the formable material layer is imprinted using at least one of: nano-imprint lithography; roll to plate lithography; roll to roll lithography; plate to plate lithography; and, plate to roll lithography.

In one embodiment the method includes micro-injection molding a plurality of microstructures shaped to breach a functional barrier of the subject.

In one embodiment the formable material includes at least one of: monomers; oligomers; photo-initiators; crosslinking acrylate groups; curable sol-gels; epoxies; resins; polymers; a curable formable material; a UV curable formable material; a sealant; a UV glue UV nano-imprint lithography polymers; Helioseal; urethane dimethacrylate; bisphenol A-glycidyl methacrylate; triethylene glycol dimethacrylate; NOA 61; Mercapto-ester*; Triallyl Isocyanurate; Inoflex RP+; PAK 01; NIF 2 (Asahi Glass Company); NIF 1 (Asahi Glass Company); and, Z Resist (t-butyl acrylate (96.5%), photoinitiator Irgacure 369 (3.5%));

In one embodiment the method includes solidifying the formable material using at least one of: heat; electromagnetic radiation; visible electromagnetic radiation; ultra-violet electromagnetic radiation; and, by curing the material.

In one embodiment the formable material has at least one of: a low viscosity; a viscosity of at least one of: between 10⁻³ Pa's and 1 Pa's; and, between of 0.4-0.05 Pa·s.

In one embodiment the cured formable material has at least one of: a high mechanical strength; a high resistivity; and, a high conductivity.

In one embodiment the substrate is at least one of: one of: substantially rigid; and, flexible; polycarbonate; polymethacrylimide (PMI); polyethylene terephthalate (PET); polycarbonate (PC); poly(methyl methacrylate) (PMMA); stainless steel; metal; polymer; and, silicon.

In one embodiment the method includes at least one of: applying at least one coating to at least part of at least some of the microstructures; applying a primer prior to applying at least one coating; and, applying a coating to formable material between at least some of the microstructures.

In one embodiment the coating includes at least one of: a conductive coating; a dielectric coating; a mechanical coating; gold; titanium titanium nitride; metal; ceramic; parylene; Poly(3,4-ethylene dioxythiophene); and, Iridium oxide (IrO₂);

In one embodiment the coating has a thickness of at least one of: at least 10 nm; at least 100 nm; at least 200 nm; at least 300 nm; at least 400 nm; at least 500 nm; about 3 μm; less than 4 μm; and, less than 5 μm;

In one embodiment the method includes applying a coating using at least one of: sputtering; electroplating; electroless plating; spin coating; thermal evaporation; vapour deposition; chemical vapour deposition; plasma enhanced chemical vapour deposition; and, ink-jet printing.

In one embodiment at least some of the microstructures are conductive microstructures and wherein the method includes creating the conductive microstructures by at least one of: applying a conductive coating to at least some of the microstructures; and, using a conductive formable material.

In one embodiment the method includes applying a dielectric coating to at least part of conductive microstructures.

In one embodiment the method includes: applying a dielectric coating over conductive microstructures; and, removing at least part of the dielectric coating to expose a surface of the conductive microstructure.

In one embodiment the microstructures include an dielectric coating extending over at least one of: part of a surface of the microstructure; a proximal end of the microstructure; at least half of a length of the microstructure; about 90 μm of a proximal end of the microstructure; and, at least part of a tip portion of the microstructure.

In one embodiment at least some of the microstructures include at least one electrode that at least one of: extends over a length of a distal portion of the microstructure; extends over a length of a portion of the microstructure spaced from the tip; is positioned proximate a distal end of the microstructure; is positioned proximate a tip of the microstructure; extends over at least 25% of a length of the microstructure; extends over less than 50% of a length of the microstructure; extends over about 60 μm of the microstructure; and, is configured to be positioned in a viable epidermis of the subject in use.

In one embodiment the method includes one of electrically isolating at least some of the microstructures by at least one of: removing material between the microstructures; selectively coating at least some of the microstructures; and, removing electrically conductive material between conductive microstructures to electrically isolate at least some of the microstructures.

In one embodiment the method includes removing material using at least one of: etching; laser ablation; plasma etching; chemical plasma etching; inductively coupled plasma etching; deep reactive ion etching; physical etching; oxygen plasma etching; reactive-ion etching; and, imprinting.

In one embodiment the formable material includes a lower layer adjacent the substrate and an upper layer incorporating the microstructures.

In one embodiment the method includes removing material by imprinting the upper layer.

In one embodiment the method includes providing electrical connections in electrical contact with at least some of conductive microstructures.

In one embodiment the electrical connections at least one of: extend through vias in the substrate; are provided on a surface of the substrate; and, are provided on a surface of the formable material.

In one embodiment the formable material includes mesas, with the microstructures extending from the mesas, and with electrical connections provided between the mesas.

In one embodiment the method includes creating microfluidic channels in a surface of at least one of: the substrate; and, the formable material.

In one embodiment the method includes creating pores in at least some of the microstructures.

In one embodiment at least some of the microstructures are at least one of: blades; ridges; needles; and, plates.

In one embodiment at least some of the microstructures at least one of: are at least partially tapered; have a cross sectional shape that is at least one of: circular; rectangular; cruciform; square; rounded square; rounded rectangular; ellipsoidal; and, at least partially hollow; have a surface that is at least partially at least one of: smooth; serrated; includes one or more pores; includes one or more raised portions; and, rough; are at least partially hollow; are porous; and, include an internal structure.

In one embodiment the microstructures include at least one of: plate microstructures; at least partially tapered plate microstructures; plate microstructures having a substantially rounded rectangular cross sectional shape; spaced apart substantially parallel plate microstructures; spaced apart rows of microstructures; pairs of spaced apart microstructures; and, groups of microstructures.

In one embodiment at least one of: at least some microstructures are angularly offset; at least some microstructures are orthogonally arranged; adjacent pairs of microstructures are orthogonally arranged; adjacent pairs of microstructures are angularly offset; pairs of microstructures are arranged in rows, and the pairs of microstructures in one row are orthogonally arranged relative to pairs of microstructures in other rows; and, pairs of microstructures are arranged in rows, and the pairs of microstructures in one row are angularly offset relative to pairs of microstructures in other rows.

In one embodiment the microstructures have a spacing that is at least one of: less than 1 mm; about 0.5 mm; about 0.2 mm; about 0.1 mm; and, more than 10 μm.

In one embodiment at least some of the microstructures have at least one of: a length that is at least one of: less than 300 μm; about 150 μm; greater than 100 μm; and, greater than 50 μm; a maximum width that is at least one of: greater than the length; about the same as the length; less than 300 μm; about 150 μm; and, greater than 50 μm; and, a thickness that is at least one of: less than 50 μm; about 25 μm; and, greater than 10 μm.

In one embodiment at least some of the microstructures have a tip that at least one of: has a length that is at least one of: less than 50% of a length of the microstructure; at least 10% of a length of the microstructure; and, about 30% of a length of the microstructure; and, has a sharpness of at least one of: at least 0.01 μm; at least 0.05 μm; at least 0.1 μm; less than 5 μm; and, about 1 μm.

In one embodiment the microstructures have a density that is at least one of: less than 5000 per cm²; greater than 10 per cm²; greater than 100 per cm²; about 25-50 per cm²; and, about 600 per cm².

In one embodiment at least some microstructures include an electrode having a surface area of at least one of: less than 0.2 mm²; about one of 0.13 mm², 0.07 mm² and 0.02 mm²; and, at least 0.01 mm².

In one embodiment the microstructures include anchor microstructures used to anchor the substrate to the subject and wherein the anchor microstructures at least one of: include anchoring structures; have a length greater than that of other microstructures; and, enter the dermis.

In one embodiment the microstructures include a material including at least one of: a bioactive material; a reagent for reacting with analytes in the subject; a binding agent for binding with analytes of interest; a probe for selectively targeting analytes of interest; a material to reduce biofouling; a material to attract at least one substance to the microstructures; a material to repel at least one substance from the microstructures; a material to attract at least some analytes to the projections; and, a material to repel at least some analytes from the projections.

In one embodiment at least some of the microstructures are coated with a coating that at least one of: modifies surface properties to at least one of: increase hydrophilicity; increase hydrophobicity; and, minimize biofouling; attracts at least one substance to the microstructures; repels at least one substance from the microstructures; acts as a barrier to preclude at least one substance from the microstructures; and, includes at least one of: polyethylene; polyethylene glycol; polyethylene oxide; zwitterions; peptides; hydrogels; and, SAMs.

It will be appreciated that the broad forms of the invention and their respective features can be used in conjunction and/or independently, and reference to separate broad forms is not intended to be limiting. Furthermore, it will be appreciated that features of the method can be performed using the system or apparatus and that features of the system or apparatus can be implemented using the method.

### BRIEF DESCRIPTION OF THE DRAWINGS

Various examples and embodiments of the present invention will now be described with reference to the accompanying drawings, in which:—

FIG. 1A is a schematic diagram of an example of a system for performing measurements on a biological subject;

FIG. 1B is a schematic underside view of an example of a patch for the system of FIG. 1A;

FIG. 2 is a flow chart of an example of a method for manufacturing a patch;

FIG. 3A is a schematic side view of an example of a substrate for using in manufacturing a patch;

FIG. 3B is a schematic side view of an example of a formable material layer applied to the substrate of FIG. 3A;

FIG. 3C is a schematic side view of an example of a mold applied to the formable material of FIG. 3B;

FIG. 3D is a schematic side view of an example of a patch formed using the formable material layer applied to the substrate of FIG. 3A;

FIGS. 3E to 3G are images of first example microstructures formed using hot embossing;

FIGS. 3H to 3J are images of second example microstructures formed using hot embossing;

FIG. 4A is a schematic side view of an example of roll to plate nano-imprinting lithography apparatus;

FIGS. 4B to 4E are images of example microstructures formed using nano-imprinting lithography;

FIG. 4F is a schematic diagram of microstructures including regions of altered properties;

FIG. 4G is a schematic diagram of a microstructure including pores formed by dissolving nano-spheres;

FIG. 4H is a schematic diagram of a microstructure including surface porosity formed by surface treatment of a cured microstructure;

FIG. 5 is a flow chart of an example of a method for coating a patch to form microstructure electrodes;

FIG. 6A is a schematic side view of an example of the patch of FIG. 3D coated with a primer;

FIG. 6B is a schematic side view of the patch of FIG. 6A coated with a conductive coating;

FIG. 6C is a schematic side view of the patch of FIG. 6B with the conductive coating selectively ablated to electrically isolate microstructures;

FIG. 6D is a schematic side view of the patch of FIG. 6C coated with a dielectric coating;

FIG. 6E is a schematic side view of the patch of FIG. 6D with the dielectric coating selectively ablated to expose the conductive coating;

FIG. 7A is a schematic side view of an example of a conductive microstructure coated with a dielectric coating;

FIG. 7B is a schematic side view of a first example of selective ablation of the dielectric coating of FIG. 7A;

FIG. 7C is a schematic side view of a second example of selective ablation of the dielectric coating of FIG. 7A;

FIG. 7D is a schematic side view of a third example of selective ablation of the dielectric coating of FIG. 7A;

FIG. 7E is an image of example microstructures coated with a dieletric coating;

FIG. 7F is an image of the microstructures of FIG. 7E with dieletric coating removed from a face of the tip;

FIG. 7G is a false colour image of the microstructures of FIG. 7F showing the exposed conductive material;

FIG. 8A is a schematic side view of an apparatus for selectively masking the patch;

FIG. 8B is an image of a selectively etched microstructure;

FIG. 8C is an image of a plate-to-plate apparatus for selectively masking a patch;

FIGS. 8D and 8E are side views of the plate-to-plate apparatus of FIG. 8C in use;

FIGS. 8F to 8L are images of examples of microstructures etched to remove parylene;

FIG. 9A is a schematic side view of a first example of a patch including electrical connection vias in the substrate;

FIG. 9B is a schematic side view of a second example of a patch including electrical connection vias in the substrate;

FIG. 9C is a schematic side view of an example of a patch including microstructures mounted on mesas with corresponding surface based electrical connections;

FIG. 9D is a schematic side view of an example of a patch including surface based electrical connections on a flexible substrate; and,

FIG. 10A is a schematic side view of an example of a plate microstructure;

FIG. 10B is a schematic front view of the microstructure of FIG. 10A;

FIG. 10C is a schematic underside view of an example of a patch including the microstructure of FIG. 10A;

FIG. 10D is a schematic perspective topside view of an example of substrate including pairs of blade microstructures of FIGS. 10A and 10B;

FIG. 10E is a schematic plan view of a grid of pairs of microstructures including electrical connections;

FIG. 10F is an image of an example of a patch including arrays of pairs of angularly offset plate microstructures;

FIG. 10G is a schematic side view of a specific example of a plate microstructure;

FIG. 10H is a schematic perspective view of the plate microstructure of FIG. 5G;

FIG. 10I is a schematic side view of an example of a pair of microstructures inserted into a subject for epidermal measurement;

FIG. 10J is a schematic side view of an example of a pair of microstructures inserted into a subject for dermal measurement;

FIG. 10K is an image of a further example of an array of microstructures;

FIG. 10L is an image of a further example of an array of pairs microstructures mounted on mesas;

FIGS. 11A to 11D are schematic side views of example steps in micromachining a master to create a mold for manufacturing the microstructures of FIG. 9C;

FIGS. 12A and 12B are schematic side views of a first example of steps in forming microstructures in a pre-patterned substrate;

FIGS. 12C and 12D are schematic side views of a second example of steps in forming microstructures in a pre-patterned substrate;

FIGS. 12E and 12F are schematic side views of example steps in selective resin curing using a shadow mask applied to the back side of a mold;

FIGS. 13A to 13C are examples of complex microstructure master geometries manufactured using wafer dicing;

FIGS. 13D to 13F are images of example microstructures formed using the master geometries of FIGS. 13A to 13C respectively;

FIGS. 14A and 14B are images of a first example of a master mold fabricated on a silicon wafer;

FIG. 14C is a schematic diagram of an example of dimensions of the micro structures for the master mold of FIGS. 14A and 14B;

FIGS. 14D to 14I are images of a second example of a master mold fabricated on a silicon wafer;

FIG. 14J is an image of an example of microstructures created using the mold of FIGS. 14D to 14I;

FIGS. 14K to 14N are further images of example microstructures created using the mold of FIGS. 14D to 14I;

### DETAILED DESCRIPTION OF THE PREFERRED EMBODIMENTS

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The terms “about” and “approximately” are used herein to refer to conditions (e.g. amounts, levels, concentrations, time, etc.) that vary by as much as 20% (i.e. +20%), especially by as much as 10%, 9%, 8%, 7%, 6%, 5%, 4%, 3%, 2% or 1% to a specified condition.

As used herein, the term “analyte” refers to a naturally occurring and/or synthetic compound, which is a marker of a condition (e.g., drug abuse), disease state (e.g., infectious diseases), disorder (e.g., neurological disorders), or a normal or pathologic process that occurs in a subject (e.g., drug metabolism), or a compound which can be used to monitor levels of an administered or ingested substance in the subject, such as a medicament (substance that treats, prevents and/or alleviates the symptoms of a disease, disorder or condition, e.g., drug, vaccine etc.), an illicit substance (e.g. illicit drug), a non-illicit substance of abuse (e.g. alcohol or prescription drug taken for non-medical reasons), a poison or toxin (including an environmental contaminant), a chemical warfare agent (e.g. nerve agent, and the like) or a metabolite thereof. The term “analyte” can refer to any substance, including chemical and/or biological agents that can be measured in an analytical procedure, including nucleic acids, peptides, enzymes, antibodies, proteins, illicit drugs, explosives, toxins, pharmaceuticals, carcinogens, poisons, allergens, and infectious agents, which can be measured in an analytical procedure. The analyte may be a compound found directly in a sample such as biological tissue, including body fluids (e.g. interstitial fluid), from a subject, especially in the dermis and/or epidermis. In particular embodiments, the analyte is a compound found in the interstitial fluid. In some embodiments, the analyte is a compound with a molecular weight in the range of from about 30 Da to about 100 kDa, especially about 50 Da to about 40 kDa. Other suitable analytes are as described herein.

As used herein, the term “and/or” refers to and encompasses any and all possible combinations of one or more of the associated listed items, as well as the lack of combinations when interpreted in the alternative (or).

As used herein, the term “aptamer” refers to a single-stranded oligonucleotide (e.g. DNA or RNA) that binds to a specific target molecule, such as an analyte. An aptamer may be of any size suitable for binding such target molecule, such as from about 10 to about 200 nucleotides in length, especially from about 30 to about 100 nucleotides in length.

The term “bind” and variations such as “binding” are used herein to refer to an interaction between two substances, such as an analyte and an aptamer or an analyte and a molecularly imprinted polymer. The interaction may be a covalent or non-covalent interaction, particularly a non-covalent interaction.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Thus, the use of the term “comprising” and the like indicates that the listed integers are required or mandatory, but that other integers are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “plurality” is used herein to refer to more than one, such as 2 to 1×10¹⁵ (or any integer therebetween) and upwards, including 2, 10, 100, 1000, 10000, 1×10⁶, 1×10⁷, 1×10⁸, 1×10⁹, 1×10¹⁰, 1×10¹¹, 1×10¹², 1×10¹³, 1×10¹⁴, 1×10¹⁵, etc. (and all integers therebetween).

As used herein, the term “predetermined threshold” refers to a value, above or below which indicates the presence, absence or progression of a disease, disorder or condition; the presence or absence of an illicit substance or non-illicit substance of abuse; or the presence or absence of a chemical warfare agent, poison and/or toxin. For example, for the purposes of the present invention, a predetermined threshold may represent the level or concentration of a particular analyte in a corresponding sample from an appropriate control subject, such as a healthy subject, or in pooled samples from multiple control subjects or medians or averages of multiple control subjects. Thus, a level or concentration above or below the threshold indicates the presence, absence or progression of a disease, disorder or condition; the presence or absence of an illicit substance or non-illicit substance of abuse; or the presence or absence of a chemical warfare agent, poison and/or toxin, as taught herein. In other examples, a predetermined threshold may represent a value larger or smaller than the level or ratio determined for a control subject so as to incorporate a further degree of confidence that a level or ratio above or below the predetermined threshold is indicative of the presence, absence or progression of a disease, disorder or condition; the presence or absence of an illicit substance or non-illicit substance of abuse; or the presence or absence of a chemical warfare agent, poison and/or toxin. Those skilled in the art can readily determine an appropriate predetermined threshold based on analysis of samples from appropriate control subjects.

The terms “selective” and “selectivity” as used herein refer to molecularly imprinted polymers or aptamers that bind an analyte of interest without displaying substantial binding of one or more other analytes. Accordingly, a molecularly imprinted polymer or aptamers that is selective for an analyte, such as troponin or a subunit thereof, exhibits selectivity of greater than about 2-fold, 5-fold, 10-fold, 20-fold, 50-fold, 100-fold or greater than about 500-fold with respect to binding of one or more other analytes.

The term “subject” as used herein refers to a vertebrate subject, particularly a mammalian subject, for whom monitoring and/or diagnosis of a disease, disorder or condition is desired. Suitable subjects include, but are not limited to, primates; avians (birds); livestock animals such as sheep, cows, horses, deer, donkeys and pigs; laboratory test animals such as rabbits, mice, rats, guinea pigs and hamsters; companion animals such as cats and dogs; bats and captive wild animals such as foxes, deer and dingoes. In particular, the subject is a human.

System for Interacting with a Subject

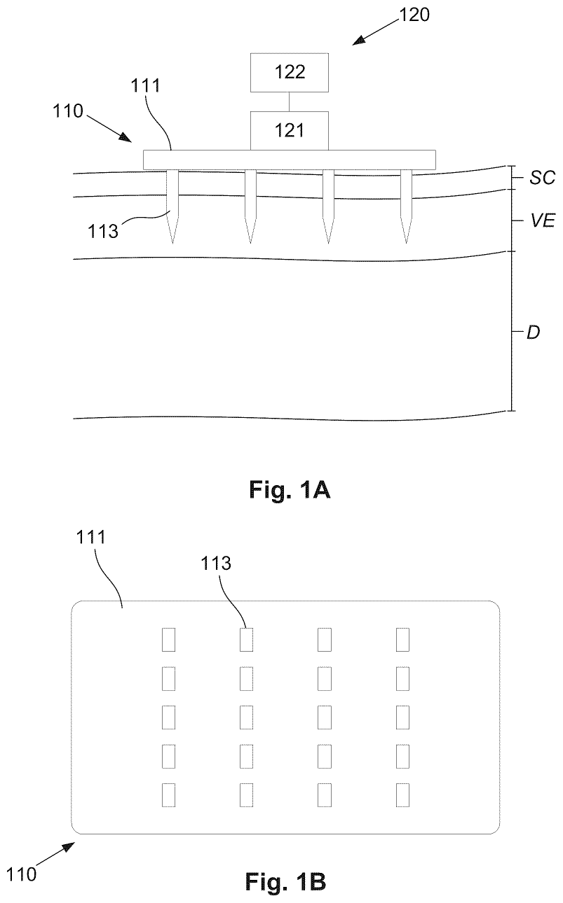

An example of a system for performing measurements on a biological subject will now be described with reference to FIGS. 1A and 1B.

In this example, the system 120 includes at least one substrate 111 having one or more microstructures 113. In use, the microstructures are configured to breach a functional barrier associated with a subject. In the current example, the functional barrier is the stratum corneum SC, and the microstructures are configured to breach the stratum corneum SC by penetrating the stratum corneum SC and entering at least the viable epidermis VE. In one particular example, the microstructures are configured to not penetrate a boundary between the viable epidermis VE and the dermis D, although this is not essential and structures that penetrate into the dermis could be used as will be described in more detail below.

Whilst this example is described with respect to breaching of the stratum corneum SC, it will be appreciated that this is not essential, and the techniques could equally be applied to other functional barriers. In this regard, a functional barrier will be understood to include any structure, boundary, or feature, whether physical or otherwise, that prevents passage of signals, and/or analytes, such as biomarkers. For example, functional barriers could include one or more layers, a mechanical discontinuity, such as a discrete change in tissue mechanical properties, a tissue discontinuity, a cellular discontinuity, a neural barrier, a sensor barrier, a cellular layer, skin layers, mucosal layers, internal or external barriers, an inner barrier within an organ, an outer barrier of organs other than the skin, epithelial layers or endothelial layers, or the like. Functional barriers could also include other internal layers or boundaries, including optical barriers such as a melanin layer, electrical barriers, molecular weight barriers that prevent passage of a biomarkers with certain molecular weights, a basal layer boundary between the viable epidermis and dermis, or the like.

The nature of the microstructure will vary depending upon the preferred implementation. In one example, the microstructures could include needles, but this is not essential and more typically structures, such as plates, blades, or the like, are used, as will be described in more detail below.

The substrate and microstructures could be manufactured from any suitable material, and the material used may depend on the intended application, for example depending on whether there is a requirement for the structures to be optically and/or electrically conductive, or the like. The substrate can form part of a patch 110, which can be applied to a subject, although other arrangements could be used for example, having the substrate form part of a housing containing other components.

In one example at least one sensor 121 is provided, which is operatively connected to at least one microstructure 113, thereby allowing response signals to be measured from respective microstructures 113. In this regard, the term response signal will be understood to encompass signals that are intrinsic within the subject, such ECG (Electrocardiogramaignals, or the like, or signals that are induced as a result of the application of stimulation, such as bioimpedance signals, or the like.

The nature of the sensor will vary depending on the preferred implementation and the nature of the sensing being performed. For example, the sensing could include sensing electrical signals, in which case the sensor could be a voltage or current sensor, or the like. Alternatively, optical signals could be sensed, in which case the sensor could be an optical sensor, such as a photodiode, CCD (Charge Coupled Device) array, or similar, whilst temperature signals could be sensed using a thermistor or the like.

The manner in which the sensor 121 is connected to the microstructure(s) 113 will also vary depending on the preferred implementation. In one example, this is achieved using connections between the microstructure(s) 113 and the sensor, with the nature of the connections varying depending upon the signals being sensed, so that the connections could include electrically conductive elements to conduct electrical signals, a wave guide, optical fibre or other conductor to conduct electromagnetic signals, or thermal conductor to conduct thermals signals. Connections could also include wireless connections, allowing the sensor to be located remotely. Ionic connections could also be used. Furthermore, connections could be provided as discrete elements, although in other examples, the substrate provides the connection, for example, if the substrate is made from a conductive plate which is then electrically connected to all of the microstructures. As a further alternative, the sensor could be embedded within or formed from part of the microstructure, in which connections may not be required.

The sensor 121 can be operatively connected to all of the microstructures 113, with connections being collective and/or independent. For example, one or more sensors could be connected to different microstructures to allow different measured response signals to be measured from different groups of microstructures 113. However, this is not essential, and any suitable arrangement could be used.

In addition to providing sensing, in some examples, the microstructures 113 could additionally and/or alternatively be configured to provide stimulation. For example, microstructures could be coupled to a signal generator that generates a stimulatory signal, as will be described in more detail below. Such stimulation could again include electrical stimulation, using a voltage or current source, optical stimulation, using a visible or non-visible radiation source, such as an LED or laser, thermal stimulation, or the like, and could be delivered via the same microstructures used for measuring response signals, or different microstructures, depending on the preferred implementation. Additionally and/or alternatively, stimulation could be achieved using other techniques, such as through exposure of the subject to the microstructures and materials thereon or therein. For example, coatings can be applied to the microstructures, allowing material to be delivered into the subject beyond the barrier, thereby stimulating a response within the subject.

These options allow a range of different types of sensing to be performed, including detecting electrical signals within the body, such as ECG signals, plethysmographic signals, electromagnetic signals, or electrical potentials generated by muscles, neural tissue, blood, or the like, detecting photoplethysmographic effects, electromagnetic effects, such as fluorescence, detecting mechanical properties, such as stress or strain, or the like. Sensing could include detecting the body's response to applied electrical signals, for example to measure bioimpedance, bioconductance, or biocapacitance, detecting the presence, absence, level or concentration of analytes, for example by detecting electrical or optical properties, or the like.

The system further includes one or more electronic processing devices 122, which can form part of a measuring device, and/or could include electronic processing devices forming part of one or more processing systems, such as computer systems, servers, client devices, or the like as will be described in more detail below. In use, the processing devices 122 are adapted to receive signals from the sensor 121 and either store or process the signals. For ease of illustration the remaining description will refer generally to a processing device, but it will be appreciated that multiple processing devices could be used, with processing distributed between the devices as needed, and that reference to the singular encompasses the plural arrangement and vice versa.

An example of the manner in which this is performed will now be described.

In particular, in one example, the substrate is applied to the subject so that the one or more microstructures breach, and in one example, penetrate the functional barrier. For example, when applied to skin, the microstructures could penetrate the stratum corneum and enter the viable epidermis as shown in FIG. 1A. This could be achieved manually and/or through the use of an actuator, to help ensure successful penetration.

Response signals within the subject are measured, with signals indicative of the measured response signals being provided to the electronic processing device 121. This may be performed following application of stimulation, although this is not essential and will vary depending on the nature of the sensing being performed.

The one or more processing devices then either analyse the resulting measurement data, and/or store the data based on the measurement data for subsequent analysis, or could alternatively provide an output based on the measured response signals. For example, the processing device could display an indicator indicative of measured response signals and/or values derived therefrom. Alternatively, the processing device could generate a recommendation for an intervention, trigger an action, such as alerting a clinician, trainer or guardian, or the like.

The analysis can be performed in any suitable manner, and this will vary depending on nature of the measurements being performed. For example, this could involve examining measured response signal values and using these to calculate an indicator indicative of a health status, including the presence, absence, degree or prognosis of one or more medical conditions, a prognosis associated with a medical condition, a presence, absence, level or concentration of a biomarker, a presence, absence, level or concentration of an analyte, a presence, absence or grade of cancer, fluid levels in the subject, blood oxygenation, a tissue inflammation state, bioelectric activity, such as nerve, brain, muscle or heart activity, or a range of other health states. This could be achieved by monitoring changes in the values over time, and may involve comparison to values measured for reference subjects having known medical conditions. Additionally, and/or alternatively, the indicator could be indicative of measured parameters associated with the subject, such as measured, level concentrations of analytes or other biomarkers.

For example, when measuring fluid levels, this could involve examining the applied stimulatory signals and values of the measured response signals, using these to calculate a bioimpedance within the epidermis, which in turn allows an indicator indicative of fluid levels to be derived. In this regard, it will be understood that fluids within the body, such as interstitial fluid, contains ions, such as Sodium (Na+), Potassium (K+), Calcium (Ca²+), Chloride (Cl—), Bicarbonate (HCO₃—) and Phosphate (HPO₄²—). As fluid levels increase or decrease, for example as the subject's level of hydration increases or decreases, there will be a corresponding fall or rise in ion concentrations, thereby resulting in a change in conductivity of the fluid. Accordingly, measuring the impedance of the fluid can in turn be used to derive information regarding fluid conductivity, which is in turn indicative of ion concentrations and hence fluid levels. Thus, it will be appreciated that this allows changes in impedance to be used to track changes in fluid levels and hence a hydration state of the subject. Such fluid levels could include any one or more of interstitial fluid levels, a change in interstitial fluid levels, an ion concentration in interstitial fluid, a change in an ion concentration in interstitial fluid, an ion concentration, a change in an ion concentration, a total body water, intracellular fluid levels, extracellular fluid levels, plasma water levels, fluid volumes or hydration levels.

The fluid level indicator could then be used in monitoring a health status, such as hydration levels, and/or a presence, absence, degree or prognosis of one or more medical conditions, a prognosis associated with a medical condition, or the like. This could also involve monitoring changes in the values over time, for example to perform longitudinal hydration measurements, and may involve comparison to values measured for reference subjects having known hydration levels, thereby allowing an assessment to be made as to whether the subject is under or over hydrated.

In any event, it will be appreciated that the above described system operates by providing microstructures that are configured to breach a barrier, such as the stratum corneum, allowing these to be used to measure response signals within the subject, such as within the epidermis and/or dermis. These response signals can then be processed and subsequently analysed, allowing a variety of values to be derived, which could be indicative of specific measurements, or one or more aspects of subject health.

For example, the system can be configured to measure an analyte level or concentration, such as the level or concentration of a specific biomarker. Response signals could also be used to generate a visualization, a spatial mapping in 1, 2 or 3 dimensions, details of mechanical properties, forces, pressures, muscle movement, blood pulse wave, an analyte concentration such as the presence, absence, level or concentration of specific biomarkers, a blood oxygen saturation, a bioimpedance, a biocapacitance, a bioconductance or electrical signals within the body, such as ECG (Electrocardiogramaignals.

In one example, the system can be configured so that measurements are performed at a specific location within the subject, such as within the epidermis only, the dermis only, or the like. This allows targeted analyte detection to be performed with a high level of accuracy, providing higher quality data for more precise measures of analytes. Furthermore, constraining the location in which measurements are performed ensures these are repeatable, allowing for more accurate longitudinal monitoring.

In contrast to traditional approaches, breaching and/or at least partially penetrating a functional barrier, such as the stratum corneum, allows measurements to be performed from within or under the barrier, and in particular within the epidermis and/or dermis, resulting in a significant improvement in the quality and magnitude of response signals that are detected. In particular, this ensures that the response signals accurately reflect conditions within the human body, and in particular within the epidermis and/or dermis, such as the presence, absence, level or concentration of biomarkers, the impedance of interstitial fluid, or the like, as opposed to traditional external measurements, which are unduly influenced by the environment outside the barrier, such as the physical properties of the skin surface, such as the skin material properties, presence or absence of hair, sweat, mechanical movement of the applied sensor, or the like. Additionally, by penetrating the stratum corneum but not the dermis, this allows measurements to be constrained to the epidermis only, thereby avoiding interference from fluid level changes in the dermis.

For example, this allows accurate measurement of high molecular weight biomarkers to be performed, which would otherwise only pass through the skin poorly. A good example of this, is glucose, which whilst present externally, such as in sweat, is typically only present in low concentrations, and often time delayed, meaning the concentration in sweat does not necessarily reflect current glucose levels within the body. In contrast, by breaching the barrier, in this case the stratum corneum, this allows far more accurate measurements to be performed. It will be appreciated that similar considerations apply to a wide range of different biomarkers or signals, and associated barriers that otherwise prevent accurate measurement of the biomarkers or signals.

For example, in the case of impedance measurements microstructure electrodes tend to measure different impedances as opposed to standard surface electrodes, which is indicative of the fact that the microstructure electrodes do not measure surface skin impedance, meaning the measured impedance is more indicative of conditions within the body and fluid compartments such as intracellular fluid and extracellular fluid compartments. As the contribution of the skin surface impedance is significant in magnitude this can result in changes in impedance within the body being masked, meaning skin based measurements are less likely to be able to detect meaningful changes.

Additionally, in some examples, the microstructures only penetrate the barrier a sufficient distance to allow a measurement to be made. For example, in the case of skin, the microstructures are typically configured to enter the viable epidermis and not enter the dermal layer. This results in a number of improvements over other invasive techniques, including avoiding issues associated with penetration of the dermis, such as pain caused by exposure of nerves, erythema, petechiae, or the like. Avoiding penetrating the dermal boundary also significantly reduces the risk of infection, allowing the microstructures to remain embedded for prolonged periods of time, such as several days, which in turn can be used to perform longitudinal monitoring over a prolonged time periods. However, in some instances, such as when detecting troponin or a subunit thereof, penetration of the dermal barrier may be required.

It will be appreciated that the ability of the microstructures to remain in-situ is particularly beneficial, as this ensures that measurements are made at the same site within the subject, which reduces inherent variability arising from inaccuracies of replacement of measuring equipment which can arise using traditional techniques. Despite this, it will be appreciated that the system can be used in other manners, for example to perform single time point monitoring or the like.

In one example, this allows the arrangement to be provided as part of a wearable device, enabling measurements to be performed that are significantly better than existing surface based measurement techniques, for example by providing access to signals or biomarkers that cannot otherwise pass through the barrier, but whilst allowing measurements to be performed whilst the subject is undergoing normal activities and/or over a prolonged period of time. This in turn enables measurements to be captured that are more accurately reflective of the health or other status of the subject. For example, this allows variations in a subject's condition during a course of the day to be measured, and avoids measurements being made under artificial conditions, such as within a clinic, which are not typically indicative of the actual condition of the subject. This also allows monitoring to be performed substantially continuously, which can allow conditions to be detected as they arise, for example, in the case of myocardial infarction, cardiovascular disease, vomiting, diarrhoea, or similar, which can allow more rapid intervention to be sought.

The above described system can be applied to any part of the body, and hence could be used with a wide range of different functional barriers. For example, the functional barrier could be an internal or external barrier, a skin layer, a mucosal layer, an inner barrier within an organ, an outer barrier of an organ, an epithelial layer, an endothelial layer, a melanin layer, an optical barrier, an electrical barrier, molecular weight barrier, basal layer or the stratum corneum. Thus, the microstructures could be applied to the buccal mucosa, the eye, or another epithelial layer, endothelial layer, or the like. The following examples will focus specifically on application to the skin, with the functional barrier including some or all of the stratum corneum, but it will be appreciated that this is intended to be illustrative and is not intended to be limiting.

### Patch Manufacture

Typically the patch, and optionally, the associated electronics, are disposable. It will be appreciated from this that it is desirable for the patches to be manufactured in a manner that is cheap and sustainable. Despite this, as breaching of the functional barrier can influence the measurements performed, it is important that the patch and particularly the microstructures are manufactured in a manner that ensures consistency in configuration, as well as having desired functional parameters, such as conductivity, durability or strength, sharpness, or the like.

Whilst silicon etching has traditionally been used to manufacture microstructure patches, this approach is very expensive and impractical for high volume manufacture. Accordingly, in a preferred example, a method for manufacturing a patch is provided which includes using a formable material to form a plurality of microstructures on a substrate, with the microstructures being shaped to breach a functional barrier of the subject and then solidifying the formable material.

Such approaches have a number of benefits. For example, forming the microstructures from formable materials allow different techniques to be used to create the microstructures, including imprinting (UV and/or thermal) or hot embossing, as well as molding. In each case, these allow for the patches to be manufactured cheaply and at high volumes, making the resulting devices readily available.

Additionally, the formable material can be configured to ensure the microstructures are of sufficient strength to breach and optionally penetrate the functional barrier, and are biologically inert, so that these do not interfere with or irritate the subject.

The formable material can be impregnated with materials, allowing these to modify properties of the microstructures, for example to ensure these are conductive, and/or be used to deliver material to the subject. Other examples include modifying mechanical properties, porosity, surface properties such as hydrophobicity, hydrophobicity, surface charge and zeta potential. Additionally, this can be used to functionalise the microstructures, making them capable of detecting specific analytes, allowing the microstructures to be used for a wide range of different applications.

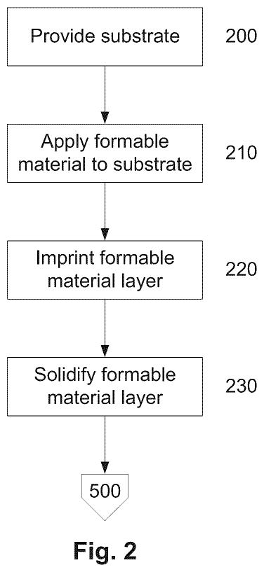

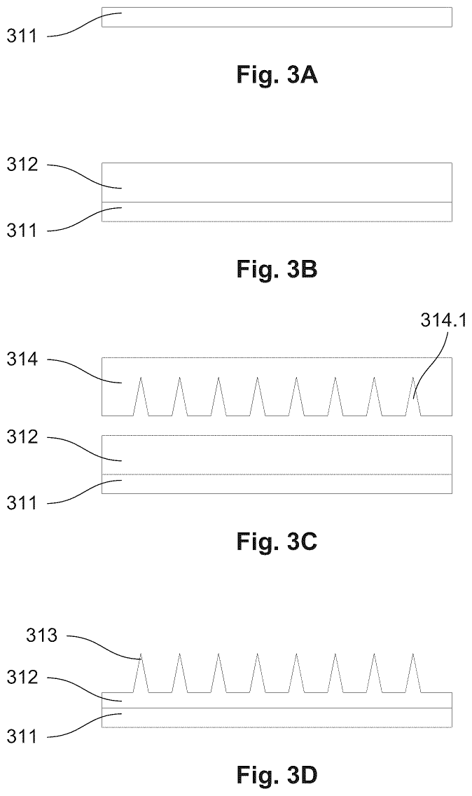

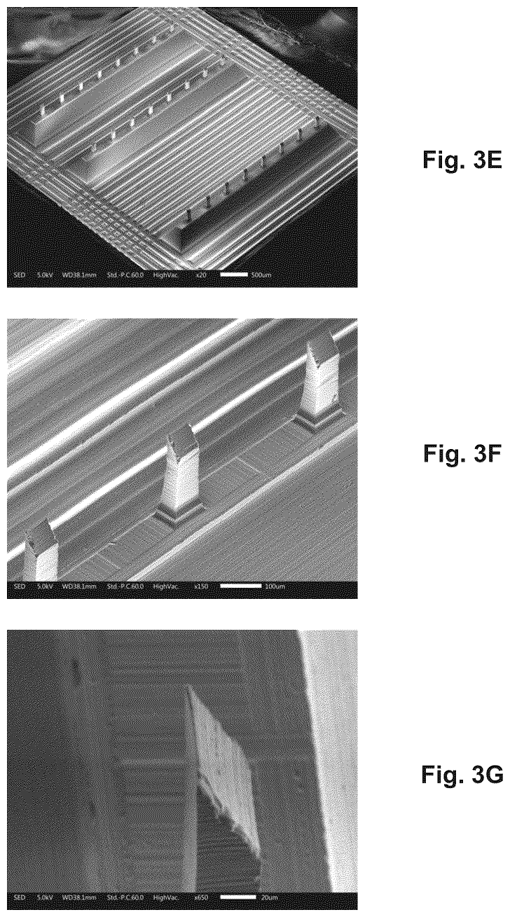

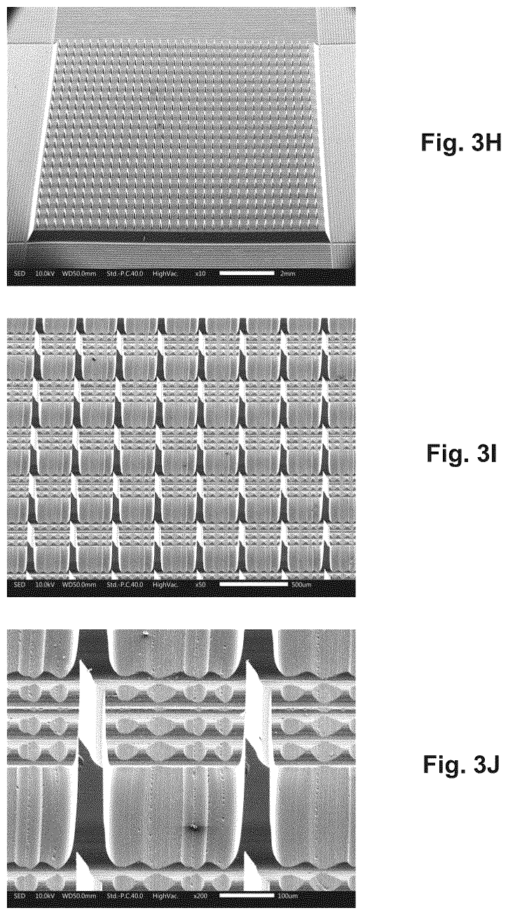

A specific example of an approach for forming the microstructures will now be described with reference to FIGS. 2 and 3A to 3D.

In this example, at step 200 a substrate 311 is provided. The substrate could be of any appropriate form, and could be a rigid, semi-rigid or flexible material, and specific examples will be described in more detail below.

A formable material layer 312 is then deposited on the substrate 311 at step 210, for example by using spraying, inkjet printing, drop casting, spin coating, doctor blading or the like. As part of this process, a surface treatment might be applied to the substrate 311 to prepare the surface and assist the formable material layer 312 bond to the substrate. A variety of surface pre-treatments could be used, including coating the substrate with a primer, for example by spraying the patch with a primer solution and/or by using a plasma pre-treatment, such as an oxygen plasma, argon plasma, or similar, for surface cleaning and/or surface activation. Other treatments could also be applied such as treating the surface with ozone.

A plurality of microstructures 313 are formed in the formable material layer 312 at step 220. In one example, this is achieved using a mold 314 having microstructure shaped cavities 314.1, which is used in an imprinting and/or hot embossing approach. The imprinting approach could use UV imprinting, thermal imprinting, or a combination of both UV and thermal imprinting, depending on the preferred implementation. Accordingly, the formable material is typically a low viscosity formable material, allowing this to fill the mold, whilst the solidified formable material typically has a relatively high mechanical strength, and optionally a high resistivity and/or a high conductivity, depending on the application. Example formable materials will be described in more detail below. The cavities 314.1 are typically smooth sided to facilitate removal of the mold, although this is not essential, and surface features might be provided, for example to create porous or undulating microstructures, to thereby increase microstructure surface areas.

Following the embossing/imprinting step, the formable material is solidified at step 230, for example by curing the formable material using visible or non-visible electromagnetic radiation, heat or the like, depending on the particular formable material used.

In the case of hot embossing this is typically achieved by stamping a pattern into a polymer softened by raising the temperature of the polymer to above its glass transition temperature. A wide variety of polymers have been successfully hot embossed with micron-scale (and below) size features, including polycarbonate and PMMA. Examples of microstructures formed using hot embossing are shown in FIGS. 3E to 3J.

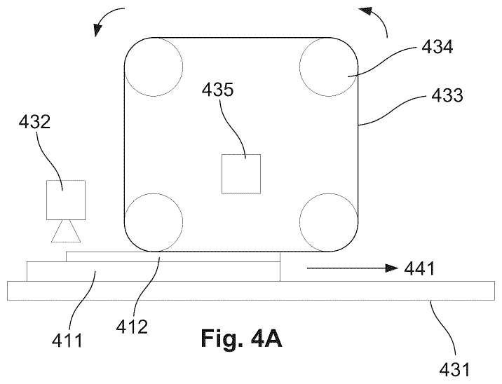

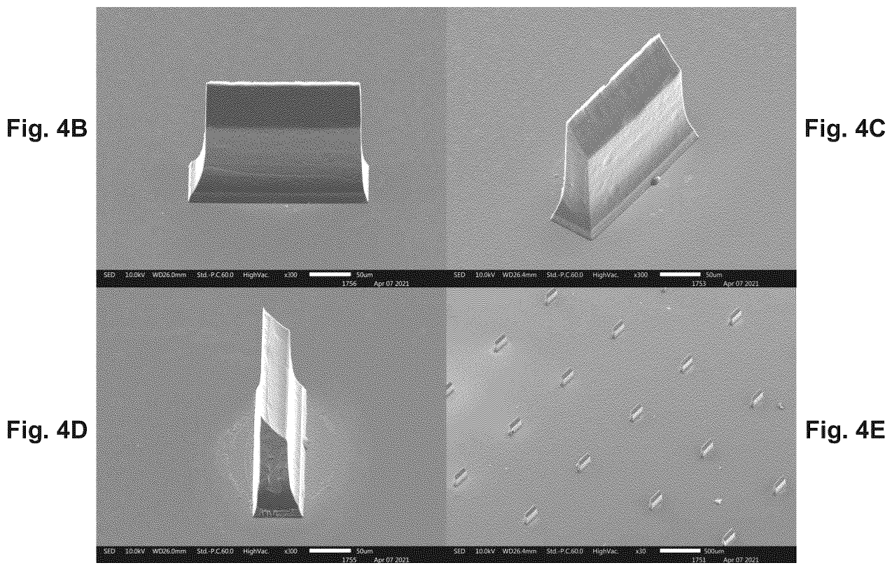

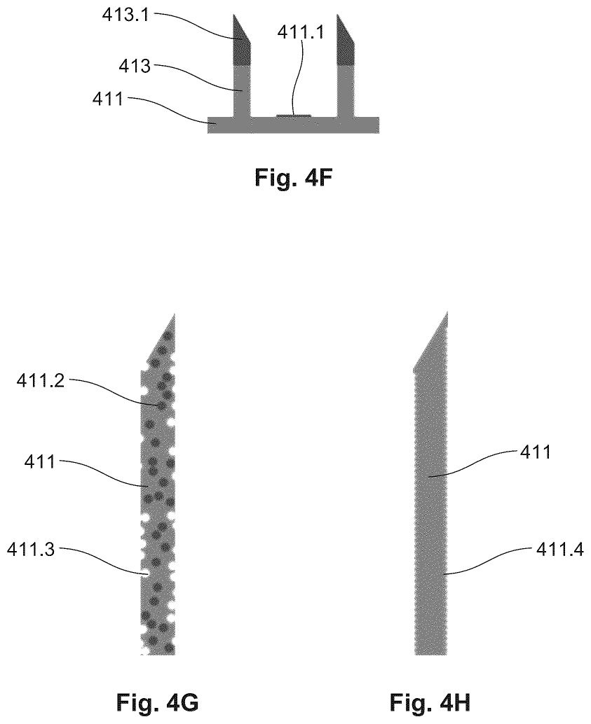

When imprinting is used, the formable material layer can be imprinted using nano-imprint lithography, roll-to-plate lithography, roll-to-roll lithography, plate-to-plate lithography and or plate-to-roll lithography. In one preferred example, roll-to-plate lithography is used and an example of this will now be described with reference to FIG. 4A.

In this example, the imprinting is performed using an apparatus including a movable support 431, such as a conveyor belt or similar, which receives the substrate 411. The substrate is transported past a formable material applicator 432, which deposits formable material onto the substrate 411, typically in one or more layers 412. The apparatus further includes an endless belt 433 entrained around rollers 434, and arranged to so that the belt is urged into engagement with the formable material layer 412 as the substrate moves in the direction of arrow 441. The belt surface incorporates the mold, so that the formable material layer is molded during the engagement. A curing device, such as a heat or radiation source 435 is provided so that the microstructures are cured, before being removed from the mold as the substrate passes out from under the belt. Example systems for imprinting in this manner include Morphotronics Portis imprinting machine. However, it will also be appreciated that other approaches could be used for creating the microstructures, such as micro-injection molding, or the like.

Examples of microstructures formed using this approach are shown in FIGS. 4B to 4E.

The mold 314 can incorporate one or more materials, coatings or other additives, either within the body of the microstructure, or through addition of a coating containing the additive. The nature of the material or additive will vary depending on the preferred implementation and could include an anti-stiction coating, such as a low surface energy material to enable easy demolding or releasing of mold 314 from the formable material 312 that is bonded to the substrate. This coating or additive could be a material or a self-assembled monolayer coating to repel the formable material or any residues left after curing. Example materials include 1H, 1H,2H,2H-Perfluorodecyltrichlorosilane, diamond-like carbon (DLC), fluorine-doped diamond-like carbon (F-DLC) film, perfluorooctyl-trichlorosilane (PFOTCS), chlorosilanes, fluorosilanes, mold material with fluorinated additives, OPTOOL from DAIKIN etc. The coating could be deposited onto the mold from vapour phase or liquid phase. It will also be appreciated that other coatings could be applied to the mold so that material is incorporated into or applied to a surface of the resulting microstructures, depending on the preferred implementation.

The anti-stiction coating can be applied to mold 314 by one and/or more methods, such as spin coating, vapour phase coating, liquid phase coating.

In spin coating, the anti-stiction material is applied to the mold 314 at a given speed and residence time to cover the entire surface area of the mold with the anti-stiction material. This method enables uniform and homogeneous coating. The resulting films do not necessarily form a monolayer, however, bestows the mold surface with low surface energy for easy demolding after imprinting.

In vapour-phase coating, anti-stiction material is applied to create monolayer on the mold surface. The mold surface needs to be activated by appropriate surface treatment procedures prior to vapour-phase coating. The coating is performed by placing the mold and the anti-stiction material inside a desiccator under low pressure or vacuum. Under such conditions, the vapours of the anti-stiction material thus produced, attach themselves to the activated surface of the mold forming a monolayer. The same can be performed in a dedicated SAM coating equipment.

In liquid phase coating of the anti-stiction material, the mold is submerged into a vessel containing the anti-stiction material. After leaving the mold anywhere between a few minutes to hours, a thin coating is formed on the mold surface. Depending on the material properties of the mold and the anti-stiction material, the coating can result in a monolayer formation or a polymer thin film and bestows the surface with low surface energy.

In another example, a mold material can be used that inherently has low surface energy properties or add an additive (that offers low surface energy) to the former at the time of making the mold.

Without an appropriate anti-stiction material (coating) or a mold material with appropriate low surface energy properties, any imprinting on the formable material can damage the mold, and/or soil the imprinted substrate and thereby render them potentially unfit for further use.

To facilitate filling of the cavities in the mold, and thereby ensure that the resulting microstructures have a desired shape, the formable material can have a low viscosity, such as between 10⁻³ Pa's and 10⁻¹ Pa's or between of 0.40-0.05 Pa·s. Filling of mold cavities can also be assisted in other manners, such as performing the molding process in a low pressure environment and/or vacuum, to thereby remove air from mold cavities. Similarly, an aperture can be provided in the mold cavity, allowing air to be ejected from, or sucked out of, the mold cavities as the imprinting process is performed.

In one example, the formable material used includes one or more of monomers, oligomers, photo-initiators, crosslinking acrylate groups, curable sol-gels, epoxies, resins, polymers, a curable formable material such as a UV curable formable material, thermal curable formable material, a sealant, a UV glue, a UV nano-imprint lithography polymer, conductive polymers, Helioseal, urethane dimethacrylate, bisphenol A-glycidyl methacrylate, triethylene glycol dimethacrylate, NOA 61, Mercapto-ester*, Triallyl Isocyanurate, Inoflex RP+, PAK 01, NIF 2 (Asahi Glass Company), NIF 1 (Asahi Glass Company), Z Resist (t-butyl acrylate (96.5%), or includes photoinitiator Irgacure 369 (3.5%)), although it will be appreciated that other suitable formable materials could be used. When using resins, these can be either a solvent-based or a solvent-free formulation. In one example discussed below, a solvent-free resin has been trialed, specifically using MM series resins from Morphotonics™ containing an acrylic backbone, such as MM1043C, MM2138B2, MM2017A, MM2394G, MM1158 or MM1158A. However, there are also solvent-based and solvent-free formulations from other resin manufacturers-microresist technology GmbH, DELO adhesives, DAIKIN Chemicals etc. that can be used. mr-NIL210SF series, OrmoClearFX, OrmoClear30 from microresist technology GmbH are other potential options. Depending on the resin formulation-solvent-based or solvent-free, a complementing porous or non-porous intermediate polymer stamp (IPS) needs to be used.

The formable material could have different properties, and could for example be hydrophobic or hydrophilic, depending on whether it is desired to attract or repel fluid. For example, a base formable material layer on the patch could be hydrophobic to repel sweat from a surface of the patch, whilst microstructures could be hydrophilic to assist with coating. It will be appreciated that different formable materials could therefore be applied in different regions of the substrate, for example using hydrophilic formable materials to construct rows of microstructures, with hydrophobic formable material being provided between the rows. Alternatively, formable materials could be provided in layers, for example forming microstructures in an upper formable material layer, whilst a lower formable material layer is provided on the substrate. An example of this is shown in FIG. 4F, in which areas of increased hydrophilicity or hydrophobicity are shown on microstructure tips 413.1 and on part of the substrate 411.1.

The formable material can include additives to modify the physical properties of the formable material, for example, including additives to increase mechanical strength, introduce porosity, or the like. Additionally, and/or alternatively, the formable material can be manipulated post curing, for example to introduce surface features, or structures, such as pores or undulations, to thereby alter the structural strength or surface area of the formable material or microstructures. This can also be performed to introduce fluid channels, for example to facilitate sweat removal, or fluid sampling, as described in more detail below.

In the example shown in FIG. 4G, a formable material is used incorporating nanospheres of polystyrene 411.2, which that are then dissolved in solvents, to yield microstructures 411 including nano-pores 411.3. Conversely, in the example of FIG. 4H, the microstructure 411 is treated after curing to introduce surface porosity 411.4.

As mentioned above, primers could be used to help the formable material adhere to the substrate, so for example, for NIL example primers include Prim1-ARK, Prim2-ARK, Prim3-ARK, and Prim4-ARK (developed by ARKEMA, doi: 10.1117/12.2515607). Another popular choice is (hexamethyldisilazane) HMDS, which can be used as a wet coating or applied from a vapor phase. One example for a commercial primer formulation based on HMDS is the mr-Primer 80/20 from microresist (https://www.microresist.de/en/produkt/mr-primer-80-20/).

In one example, the method includes solidifying the formable material for example by curing using heat, electromagnetic radiation, visible electromagnetic radiation or ultra-violet electromagnetic radiation, although again other suitable curing approaches could be used depending on the preferred implementation and the nature of the formable material. Additionally, curing parameters can be controlled to optimize the properties of the resulting microstructures, for example to ensure sufficient mechanical strength, without making the microstructures too brittle. Example parameters that can be controlled include pressure, air pressure, temperature, duration or radiation exposure dose, roller speed, roller temperature, plate speed, plate temperature, exposure time, exposure power, or the like.

The nature of the substrate will vary depending upon the preferred implementation. For example, the substrate could be made from or contain fabric, woven fabric, electronic fabric, natural fibres, silk, organic materials, natural composite materials, artificial composite materials, ceramics, stainless steel, ceramics, metals, such as stainless steel, titanium or platinum, polymers, such as rigid or semi-rigid plastics, including doped polymers, silicon or other semiconductors, including doped semiconductors, organosilicates, gold, silver, carbon, carbon nano materials, or the like. Specific example materials include polycarbonate, polymethacrylimide (PMI), polyethylene terephthalate (PET), polycarbonate (PC), poly(methyl methacrylate) (PMMA). In another example, nano-imprint lithography can also be used to define microstructures directly onto printed circuit boards (PCBs), such as thin flexible PCBs, which typically includes a metallic layer of traces, usually copper, bonded to a dielectric layer, such as polyimide.

In one example, the method includes applying at least one coating to at least part of at least some of the microstructures. Coatings can be applied using a variety of techniques, including sputtering, electroplating, electroless plating, spin coating or ink-jet printing, although other approaches such as dip coating, spray coating, deposition coating, electropolymerisation, drop casting, or the like, could also be used.

The coating typically includes a conductive coating, a dielectric coating and/or a mechanical coating. Specific example coatings include gold, silver, titanium, titanium nitride, metal, ceramic, parylene, Poly(3,4-ethylene dioxythiophene), or Iridium oxide (IrO₂), although it will be appreciated that other suitable coatings could be used.

The coating can have a thickness of at least 100 nm, at least 200 nm, at least 300 nm, at least 400 nm, about 500 nm, less than 3 μm, less than 4 μm, or less than 5 μm, although other thicknesses could be used depending on the function of the coating and the intended use.

The coating could be applied to the entire microstructures and optionally also the formable material layer and/or substrate, and/or could be applied to part of the microstructures, such as the tip or base, or could be applied and removed so that the microstructures are only partially coated. Material can be removed using a variety of approaches such as ablation, selective plasma or chemical etching, oxygen plasma etching, or the like, as will be described in more detail below, and the technique used will depend on the nature of the material being removed and the preferred implementation. For example, etching can be easier for bulk treatment, but has limitations in removing some materials, such as parylene, so in some circumstances laser ablation might be preferred.

Coatings can also be applied in multiple layers, for example to provide a functional coating, and an overlying protective coating, which is configured to dissolve on contact with fluid within the subject, to thereby expose the functional coating.

In one example, at least some of the microstructures are conductive microstructures and the method includes creating the conductive microstructures by applying a conductive coating to at least some of the microstructures and/or using a conductive formable material. The conductive microstructures can act as electrode, which could be used to apply electrical signals to a subject, measure intrinsic or extrinsic response electrical signals, for example measuring ECG or impedances. In another example, the microstructures interact with one or more analytes of interest such, with the electrodes being used to detect a response signal that is dependent on a presence, absence, level or concentration of one or more analytes of interest, thereby allowing the level or concentration of one or more analytes to be quantified.

Additionally, and/or alternatively, a dielectric coating can be applied to selectively insulate at least some of the microstructures, for example applying a dielectric coating to at least part of a conductive microstructures, so that only selected parts of the conductive microstructures are exposed. Thus, the microstructure could include an electrically conductive material covered by a non-conductive (insulating) layer, with openings providing access to the conductive material to allow conduction of electrical signals through the openings to thereby define electrodes.

In one example, when an insulating layer is used, this extends over part of a surface of the microstructure, including a proximal end of the microstructure adjacent the substrate. The insulating layer could extend over at least half of a length of the microstructure and/or about 60 μm, 90 μm or 150 μm of a proximal end of the microstructure, and optionally, at least part of a tip portion of the microstructure. In one specific example, this is performed so the non-insulating portion defining the electrode is provided in the epidermis and/or dermis, so stimulatory signals are applied to and/or response signals received from, the epidermis and/or dermis. In this instance, the electrode could extend over a length of a distal portion of the microstructure or a portion of the microstructure spaced from the tip. The electrode can be positioned proximate a distal end of the microstructure or positioned proximate a tip of the microstructure, depending on the intended application. The electrode can extend over at least 25% of a length of the microstructure, less than 50% of a length of the microstructure, or about 60 μm of the microstructure.

In one example, such arrangements are created by applying a dielectric coating over conductive microstructures and then removing at least part of the dielectric coating to expose the conductive surface. This approach has the benefit that the entire microstructure can be conductive, making it easy to provide electrical connections to the microstructures, but that only part of the microstructure is exposed, so that the extent of the resulting electrode is controlled, which can in turn allow measurements/stimulation to be performed at a defined location within the body. However, it will be appreciated that electrodes could be defined using other approaches, such as only coating part of the microstructures with a conductive material, or removing conductive material applied to parts of the microstructures, or using a conductive forming material, optionally partially covered using a dielectric coating.

The insulating layer could also extend over some or all of a surface of the substrate. In this regard, in some examples connections are formed on a surface of the substrate, in which case a coating could be used to isolate these from the subject. For example, electrical tracks on a surface of the substrate could be used to provide electrical connections to the electrodes, with an insulating layer being provided on top of the connections to ensure the connections do not make electrical contact with the skin of the subject, or any sweat present, which could in turn adversely affect measured response signals. Electrically conductive via holes can also be provided in the substrate to be used to transfer electrical tracks to an underside of the substrate, as will be described in more detail below.

In addition to providing an electrode over only part of a microstructure, it may also be necessary to electrically isolating at least some of the microstructures, for example, allowing different signals to be applied to or measured from different rows of microstructures. In one example, this can be achieved by removing material between the microstructures, selectively coating at least some of the microstructures, or removing electrically conductive material between conductive microstructures to electrically isolate at least some of the microstructures.

In one example, the method of removing material, for example to remove either a dielectric or conductive coating can include removing material using etching, ablation, plasma etching, chemical plasma etching, inductively coupled plasma etching, deep reactive ion etching, physical etching, oxygen plasma etching, reactive-ion etching, imprinting, or the like. For example, some of the material could be masked using a chemical mask, such as photoresist, shadow mask, or a physical mask such as a foil material, allowing selective etching to be performed.

In another example, the formable material layer can include a lower layer adjacent the substrate and an upper layer incorporating the microstructures, with the material between the electrodes being removed by imprinting the upper layer. In this example, if only the upper layer is conductive, this can be used to isolate adjacent microstructures.

Coating can also be used to provide additional functionality. For example, the coating could incorporate materials, such as therapeutic materials, which with stimulation, such as chemical, biochemical, electrical, optical or mechanical stimulation, can be used to release material from the coating on the microstructure, disrupt the coating, dissolve the coating or otherwise release the coating.

In another example, the microstructures can be coated with a selectively dissolvable coating. The coating could be adapted to dissolve after a defined time period, such as after the microstructures have been present within the subject for a set length of time, in response to the presence, absence, level or concentration of one or more analytes in the subject, upon breaching or penetration of the functional barrier, or in response application of a stimulatory signal, such as an electrical signal, optical signal or the like. Dissolving of the coating can be used in order to trigger a measurement process, for example by exposing a binding agent, or other functional feature, so that analytes are only detected once the coating has dissolved.

In a further example, dissolving of the coating could be detected, for example through a change in optical or electrical properties, with the measurement being performed after the coating has dissolved. Thus, dissolving of the coating could be detected based on a change in a response signal.

In one example, the coating can be used to provide mechanical properties. For example, the coating can provide a physical structure that can be used to facilitate penetration of the barrier, for example by providing a microstructure with a smooth tapered outer profile. The coating can strengthen the microstructures, to prevent microstructures breaking, fracturing, buckling or otherwise being damaged during insertion, or could be used to help anchor the microstructures in the subject. For example, the coating could include hydrogels, which expand upon exposure to moisture, so that the size of the microstructure and coating increases upon insertion into the subject, thereby making it harder to remove the microstructure. Additionally, as well as assisting with skin engagement, gels can be used to block penetration holes and stop fluid leaking out, or sweat getting into the holes.

The coating can also be used to modify surface properties of the microstructures, for example to increase or decrease hydrophilicity, increase or decrease hydrophobicity and/or minimize biofouling. The coating can also be used to attract, repel or exclude at least one substance, such as analytes, cells, fluids, or the like. The coating could also dissolve to expose a microstructure, a further coating or material, allowing this to be used to control the detection process. For example, a time release coating could be used to enable a measurement to be performed a set time after the patch has been applied. This could also be used to provide stimulation to the subject, for example by releasing a treatment or therapeutic material, or the like.

Microstructures could be differentially coated, so that different microstructures are differentially responsive to analytes. For example, different microstructures could be responsive to different analytes, responsive to different combination of analytes, responsive to different levels or concentrations of analytes, or the like.

In one example, at least some of the microstructures attract at least one substance to the microstructures and/or repel or exclude at least one substance from the microstructures. The nature of the substance will vary depending on the preferred implementation and may include one or more analytes, or may include other substances containing analytes, such as ISF, blood or the like. This can be used to attract, repel or exclude analytes, for example attracting analytes of interest, allowing these to be concentrated and/or sensed, or repelling or excluding analytes that are not of interest.

The ability to repel or exclude substances can also assist with preventing biofouling. For example, the microstructures could contain a material, or include a coating, such as polyethylene glycol (PEG), which generally repels substances from the surface of the microstructure. Reduction in biofouling could also be achieved based on a choice of microstructure material or structure of the microstructure e.g. coating the binding agent in the pores of a porous microstructure, surface coatings that release to expose a sensing surface when sensing is to be performed, permeable coatings such as a porous polymer e.g. a nylon membrane, a polyvinylidenefluoride coating, a polyphenylenediamine coating, a polyethersulfone coating, or a hydrogel coating such as a poly(hydroxyethyl methacrylate) or PEG coating; an isoporous silica micelle membrane; a protein membrane, such as a fibroin membrane; a polysaccharide membrane, such as a cellulose membrane or a chitosan membrane; or a diol or silane membrane; releasable coatings that interfere with biofouling material; and/or porous coatings. In particular embodiments, the microstructure is porous, and the binding agent is coated in the pores of the microstructure.

The coating could also be used to allow analytes to be detected. For example, the coatings can include aptamers or molecularly imprinted polymers that respond to analytes, allowing changes in electrical signals to be used to detect analyte concentrations.

To optimise coating processes, properties of the coating can be controlled through the addition of one or more other agents such as a viscosity enhancer, a detergent or other surfactant, and an adjuvant. These ingredients can be provided in a range of different concentrations. For example, the viscosity enhancer or surfactant can form between 0% and 90% of the coating solution.

A range of different viscosity enhancers can be used and examples include methylcellulose, carboxymethylcellulose (CMC), gelatin, agar, and agarose and any other viscosity modifying agents. The solution typically has a viscosity of between 10⁻³ Pas and 10⁻¹ Pa's or between of 0.40-0.05 Pa·s. In one example, using a coating solution containing 1-2% methylcellulose, which results in suitable uniform coatings, resulting in a viscosity within the range 0.011 (1%)-0.055 (2%) Pa·s.

Similarly, a range of different surfactants can be used to modify the surface tension of the coating solution, such as any detergent or any suitable agent that decreases surface tension, and that is biocompatible at a low concentration. The solution properties are also typically controlled through the addition of one or more other agents such as a viscosity enhancer, a detergent, other surfactant, or anything other suitable material. These ingredients can be provided in a range of different concentrations. For example, the viscosity enhancer or surfactant can form between 0% and 90% of the coating solution.

In some embodiments it may be desirable to increase the binding (adhesion) of the coating to the microstructures. Accordingly, in such embodiments, a primer agent can be applied to the formable material layer and microstructures, prior to applying the coating, which increases binding of the coating to the microstructure. Suitable agents include, but are not limited to, organosilanes, silicones, siloxanes, amide and amine containing compounds, organophosphorus compounds, self-assembled monolayers or other coupling agents, such as titanium, chrome, metallic and semi-metallic wetting layers.

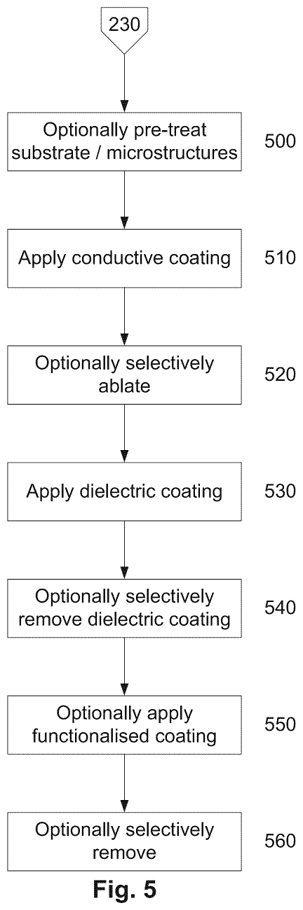

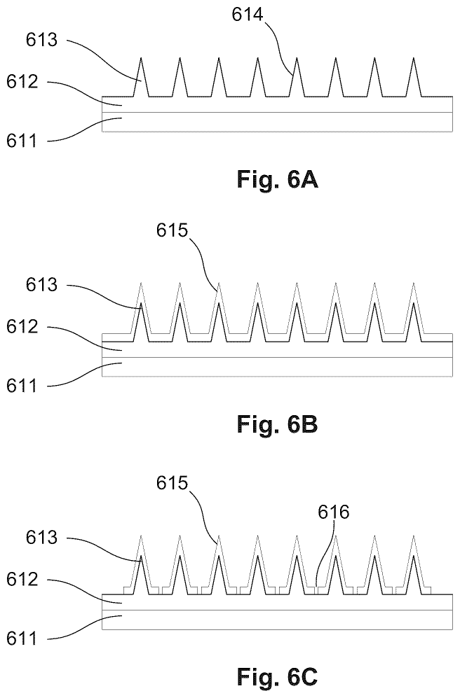

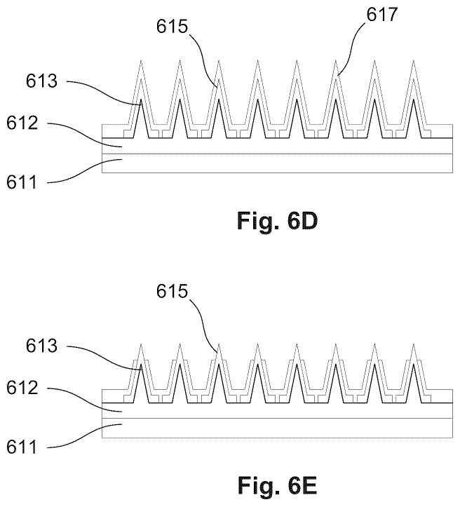

A specific example of using multiple coatings to manufacture microstructures with electrodes will now be described with reference to FIG. 5 and FIGS. 6A to 6E.

In this example, a patch is provided including a substrate 611 and a formable material layer 612 imprinted with microstructures 613. It will be appreciated that this is similar to the patch of FIG. 3D and can be manufactured using steps 200 to 230 described above, as well as performing optional steps, such as priming and/or mold coating with an anti-stiction material. The patch including the microstructures 613 is initially optionally pre-treated, for example by being optionally coated with a primer 614 at step 500, for example by spraying the patch with a primer solution and/or by using a plasma pre-treatment for surface cleaning and surface activation. Other treatments could also be applied such as treating the surface with ozone.

At step 510, a conductive coating 615 is applied to the microstructures 613 and formable material layer 612, for example using sputter coating of gold or another similar conductive material. At this stage, the entire upper surface of the patch, including all of the microstructures are conductive. However, it is typically necessary to isolate some of the microstructures, for example, isolating rows of microstructures, or isolating individual microstructure, to allow these to be used independently. Accordingly, in this example, laser ablation is used at step 520, to create openings 616 in the conductive coating, thereby electrically isolating adjacent microstructures and/or rows of microstructures.

Following this, at step 530 a dielectric coating 617 is applied to insulate the surface of the conductive coating 615. At step 540, the dielectric coating 617 is optionally selectively removed, for example using ablation or etching, in this case removing material in the region of the tips, so that the conductive coating in the tip region of the microstructure is exposed, allowing this to function as an electrode. However, it will be appreciated that this may not be required, and the dielectric coating might be retained in place, for example if the microstructure is to be used to perform capacitive sensing.

At step 550, a functionalized coating, such as an aptamers or molecularly imprinted polymer coating, can optionally be applied using inkjet printing, dip coating, spray coating or the like, depending on the intended usage of the device. Some of the functionalized coating may also be optionally removed at step 560, for example, removing functionalized coating from the substrate, or from parts of the microstructures, for example so that only tips or other portions of the microstructures are functionalized.

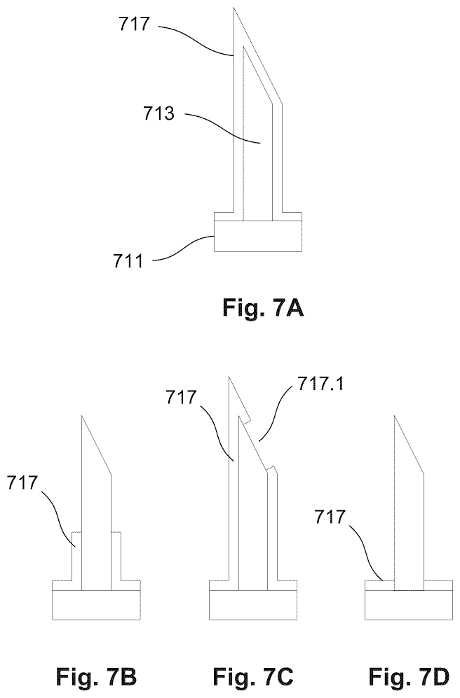

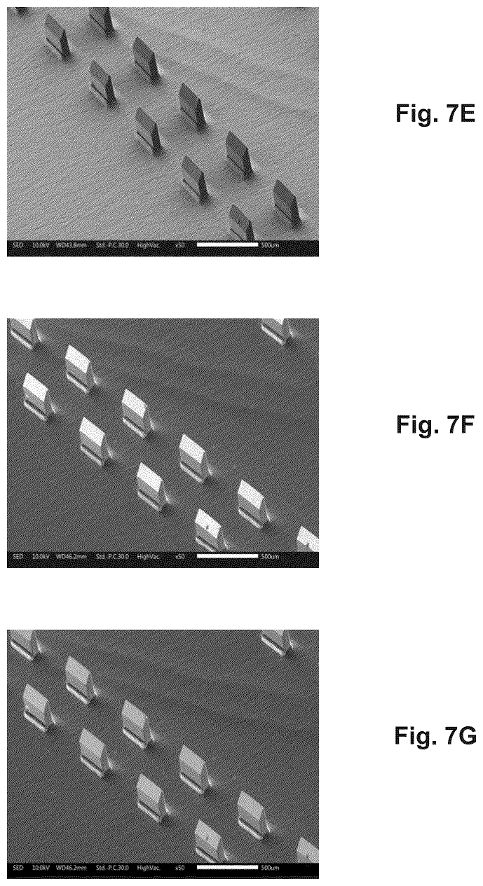

It will be appreciated that dielectric material could be removed in other locations, and an example of this will now be described with reference to FIGS. 7A to 7D.

In this example, a substrate 711 and a conductive microstructure 713 are coated with a dielectric material 717. In the example of FIG. 7B, the dielectric material 717, such as parylene is removed from a top half of the microstructure, leaving around 100 μm on the microstructure base insulated. In the example of FIG. 7C, a small section of the dielectric material 717 on the microstructure tip is removed creating an open window 717.1 on the slanted top face of the microstructure, whereas in FIG. 7D the dielectric material is removed over the entire extent of the microstructure, leaving the substrate coating untouched.

Examples of microstructures before and after etching of a dielectric coating are shown in FIGS. 7E to 7G. In this example, the coated microstructures are shown in FIG. 7E, with etched microstructures being shown in FIG. 7F, with dielectric coating removed from a face of the tips. In this instance, a false colour image is shown in FIG. 7G highlighting the exposed conductive material on the face of the tip.

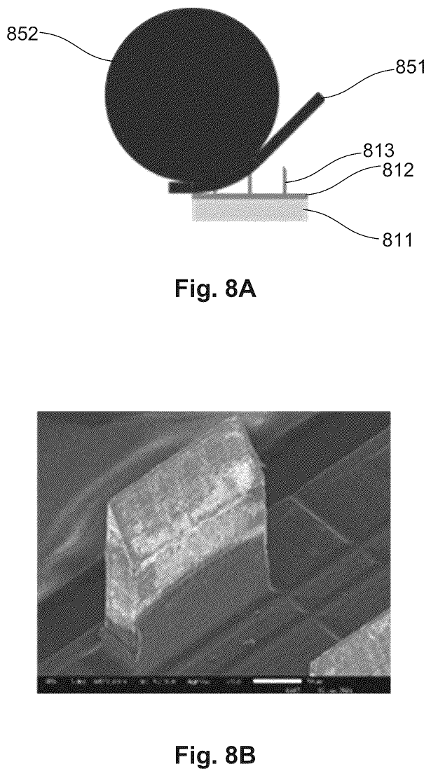

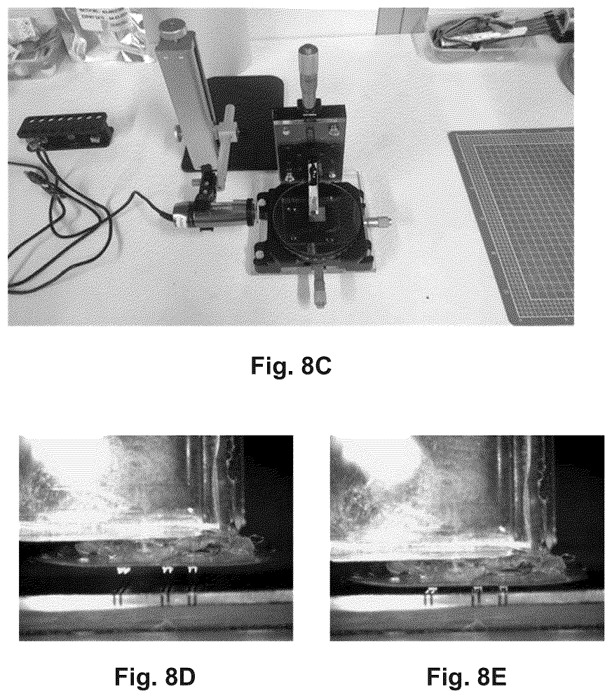

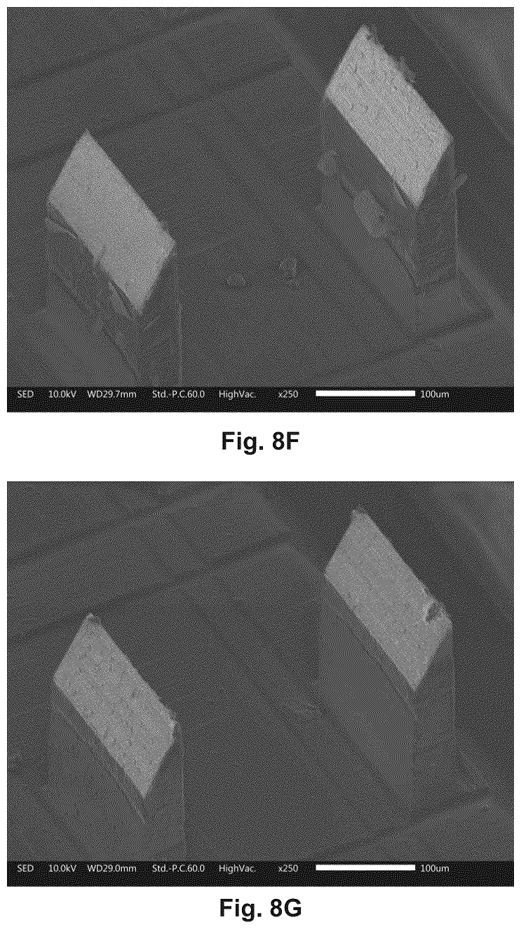

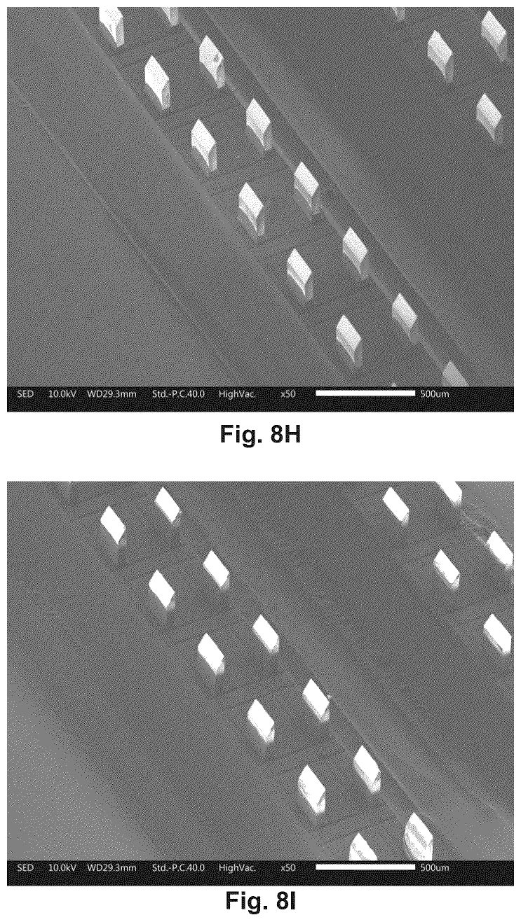

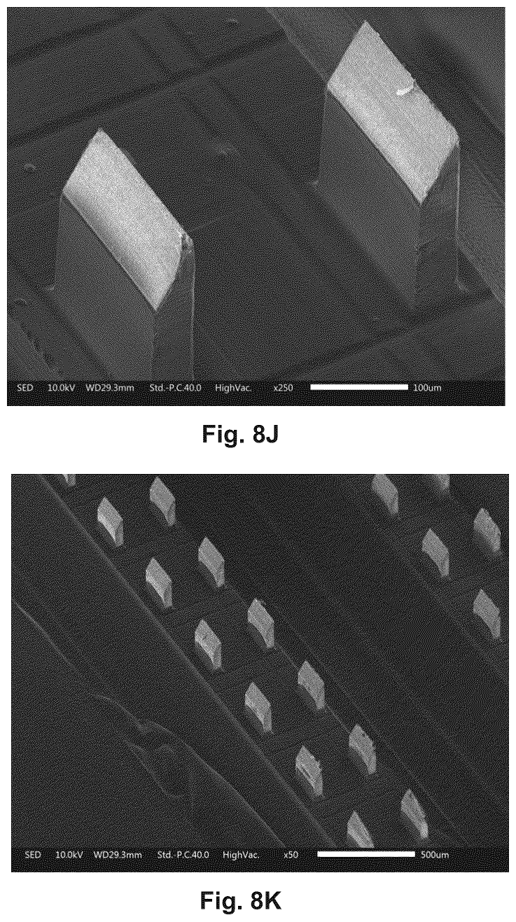

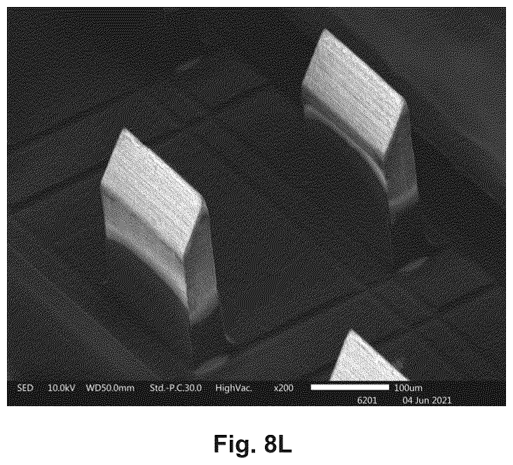

An alternative technique for removing material will now be described with reference to FIGS. 8A and 8B.

In this example, a patch including a substrate 811 having a conductive microstructure 813, formable material layer 812 and overlying dielectric layer (not shown) has a mask layer 851, such as a foil, plastic, polymer or other similar material, applied thereto using a roller 852. In one example, the roller 852 is deformable, so that the microstructures deform the roller and penetrate through the mask layer, although alternatively the roller has holes that align with the microstructures. In either case, the mask layer only covers an upper surface of the substrate and optionally also a lower part of the microstructure, so that etching can be used to remove the dielectric layer in the vicinity of the microstructure tip and thereby expose the conductive surface in the tip region as shown in FIG. 8B.

The example shown in FIG. 8A is an example of a roll-to-plate process, but roll-to-roll or plate-to-plate are also possible and FIG. 8C shows an example of a plate-to-plate foil applicator that is aligned with a substrate masked with a piece of foil (detailed below) using a microscope. As shown in FIGS. 8D and 8E, microstructures on the substrate are aligned with a matching array of grooves in a piece of silicon and then lowered into the grooves through a piece of aluminium foil to mask the base of the substrate leaving only the blade tips exposed to any subsequent plasma processing.

It will be appreciated that the masking described in FIGS. 8A to 8E may not be necessary if laser ablation or other similar approaches can be used to selectively remove materials. In particular, the mask is only needed to protect the base of the microstructure and the substrate if material cannot otherwise be selectively removed, for example if laser ablation is too slow or does not have sufficient resolution.

It will be appreciated that different etching can be used to remove different materials. For example, parylene is known to be a difficult material to remove. Oxygen plasma is the only dry etching method to remove parylene, and in this case, lateral and vertical etch rates can be controlled by varying different process parameters, such as pressure, flow rates, DC bias, or the like, as described for example in the paper “Plasma removal of Parylene C” by Ellis Meng, Po-Ying Li and Yu-Chong Tai, J. Micromech. Microeng. 18 (2008) 045004 (13pp). This allows anisotropic etching to be achieved, which in turn provides a high degree over material removal.

Other methods exist, such as mechanical removal through micro-abrasion and a chemical softening of the coating using the organic solvent tetrahydrofuran, which then allows the layer to be peeled off. Alternatively, to selectively remove parylene on a micro scale, laser ablation can be used.

FIGS. 8F to 8L are images of examples of microstructures etched to remove parylene.

In the examples of FIGS. 8F, 8G and 8J show complete parylene removal only from the slanted tip only. FIGS. 8H, 8I, 8K and 8L show microstructures with sidewalls that have also been etched/cleared. In the case of image 8I, the aluminium masking foil was sitting further up the needles, resulting in less of the needle tip getting etched.

Conversely gold can be dry-etched using different gases, such as Cl2 or a mixture of CF4 and CCl4. Ion milling is another good dry etching option.

As mentioned above, in one example, the method can include providing electrical connections in electrical contact with at least some of the conductive microstructures. This can be used to apply signals to, or measured signals from, the microstructures. The electrical connections can be provided in a variety of ways depending on the preferred implementation, and this could include vias extending through the substrate and/or surface contacts on a surface of the substrate and/or a surface of the formable material layer.

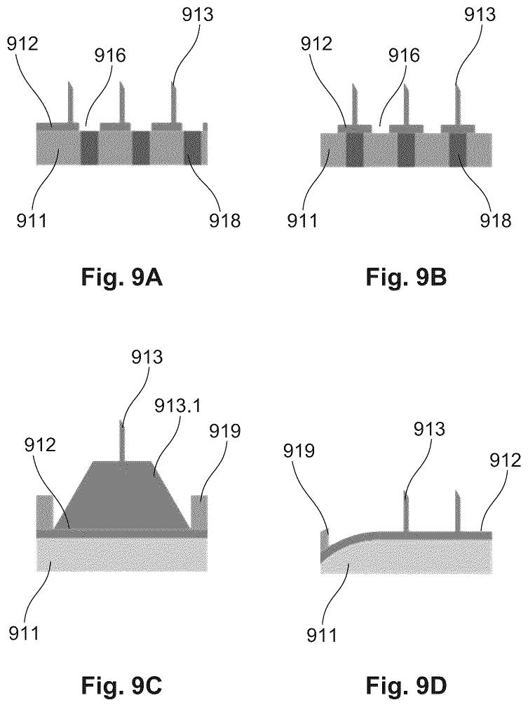

An example of this will now be described with reference to FIGS. 9A to 9D.

In the example of FIG. 9A, a patch is provided including substrate 911, formable material layer 912 and imprinted microstructures 913. Openings 916 are provided to isolate the microstructures, as described above, whilst vias 918 extend through the substrate 911 aligned with the openings 916, allowing electrical connections to be created with the microstructures 913 using a conductive layer applied to the microstructures and extending to the vias 918. This arrangement allows electrical connections to be provided on a rear surface of the substrate so that these can be easily created and do not interfere with insertion of the microstructures into the subject. Furthermore, by using a conductive coating to form an electrical connection with the vias, this allows the vias to be located offset from the microstructures and in turn avoids the need for accurate alignment of the microstructures with the vias. The microstructures can be formed on top of a substrate with preformed vias, or alternatively the vias can be formed after the microstructures, depending on the preferred implantation.

In the example of FIG. 9B, a patch is provided including substrate 911, formable material layer 912 and imprinted microstructures 913. Openings 916 are provided to isolate the microstructures, as described above, whilst vias 918 extend through the substrate 911 aligned with an underside of the microstructures 913. In this instance, assuming the formable material layer and microstructures are conductive, this allows electrical connections to be created with the underside of the formable material layer. Again this allows electrical connections to be provided on a rear surface of the substrate. Additionally, in the event that the formable material is conductive, this avoids the need to use additional conductive coatings to form an electrical connection between the vias and the microstructures.

In the example of FIG. 9C, a patch is provided including substrate 911, formable material layer 912 and imprinted microstructures 913 provided on a mesa 913.1. Electrical connections 919 are created on top of the formable material layer 912, with the mesa 913.1 supporting the microstructure 913 so that the base of the microstructure is elevated above the height of the electrical connections, thereby preventing the electrical connections interfering with penetration of the microstructures into the subject. Mesas also space the microstructures from the substrate, which can further assist in ensuring penetration of the microstructures into the subject.

In the example of FIG. 9D, a patch is provided including a flexible and/or curved substrate 911, formable material layer 912 and imprinted microstructures 913. Electrical connections 919 are created on top of the formable material layer 912, with flexing or curvature of the substrate ensuring the base of the microstructure is elevated above the height of the electrical connections, thereby preventing the electrical connections interfering with penetration of the microstructures into the subject.

In one example, the method can further include creating microfluidic channels in the substrate and/or the formable material layer. The microfluidic channels can be used to assist with removal of surface moisture, such as sweat, which may build up between the upper surface of the patch and the skin of the subject. In this instance, the channels can be provided in an upper surface of the substrate, for example prior to application of the formable material layer, with the formable material layer in the vicinity of the channels being subsequently removed by ablation, etching or the like. Alternatively, the channels can be created directly in the formable material layer using ablation or etching, or by creating the channels in the same imprinting step used to create the microstructures. In one example, the channels are formed by the cut-outs in a conductive coating, which are used to isolate different microstructures, allowing the cut-outs to serve a dual purpose.

Similarly, openings may be provided in the substrate to allow fluid to flow through to a rear surface of the substrate. In one example, the openings are offset from the microstructures, allowing surface moisture, such as sweat, to be removed to a rear surface of the patch. Alternatively, the openings can be aligned with microstructures having channels extending therethrough, for example, to allow for sampling of fluids from within the subject. In this latter example, the openings in the substrate could be pre-formed, with hollow microneedles being created using a suitable imprinting mold. Alternatively, this could be achieved by ablation of the microstructures and substrate post imprinting.

Hollow microstructures can also be used, optionally together with suitable microfluidics, to allow for extraction of fluids from the subject to allow for external sensing mechanisms to be used. For example, ISF could be extracted via hollow microneedles, undergoing testing and/or sensing on a rear side of the patch and/or externally to the patch. This can have certain benefits, such as allowing ISF to be exposed to substances that would be hazardous or undesirable in the subject. The creation of such hollow openings could be achieved in a variety of ways, including using a suitable imprinting process, or during post processing, for example using laser ablation, micro-drilling, suitable etching technique, or the like.

In one example, the method includes creating pores in at least some of the microstructures. Creating porous microstructures can increase the effective surface area of the microstructure, and can be used to help capture analytes and/or increase the effective surface area of an electrode formed on the microstructure. The pores may be of any suitable size to allow an analyte of interest to enter the pores, but exclude one or more other analytes or substances, and thus, will depend on the size of the analyte of interest. In some embodiments, the pores may be less than about 10 μm in diameter, preferably less than about 1 μm in diameter. The pores are typically created by providing an additive in the formable material which is dissolved or released after formation of the microstructures, although it will be appreciated that other techniques could be used, such as exposing the formable material to heat, or radiation, to induce bubble formation, in turn leading to porosity when the formable material cures.

The microstructures could have a range of different shapes and could include ridges, needles, plates, blades, or similar. In this regard, the terms plates and blades are used interchangeably to refer to microstructures having a width that is of a similar order of magnitude in size to the length, but which are significantly thinner. The microstructures can be tapered to facilitate insertion into the subject, and can have different cross-sectional shapes, for example depending on the intended use. The microstructures typically have a rounded rectangular shape and may include shape changes along a length of the microstructure. For example, microstructures could include a shoulder that is configured to abut against the stratum corneum to control a depth of penetration and/or a shaft extending to the tip, with the shaft being configured to control a position of the tip in the subject and/or provide a surface for an electrode.

Other example shapes include circular, rectangular, cruciform shapes, square, rounded square, rounded rectangular, ellipsoidal, or the like, which can allow for increased surface area, which is useful when coating microstructures to maximise the coating volume and hence the amount of payload delivered per microstructure, although it will be appreciated that a range of other shapes could be used. Microstructures can have a rough or smooth surface, or may include surface features, such as pores, raised portions, serrations, or the like, which can increase surface area and/or assist in penetrating or engaging tissue, to thereby anchor the microstructures within the subject. This can also assist in reducing biofouling, for example by prohibiting the adherence and hence build-up of biofilms. The microstructures might also be hollow or porous and can include an internal structure, such as holes or similar, in which case the cross-sectional shape could also be at least partially hollow. In particular embodiments, the microstructures are porous, which may increase the effective surface area of the microstructure. The pores may be of any suitable size to allow an analyte of interest to enter the pores, but exclude one or more other analytes or substances, and thus, will depend on the size of the analyte of interest. In some embodiments, the pores may be less than about 10 μm in diameter, preferably less than about 1 μm in diameter.

In one example, the microstructures have a rounded rectangular shape when viewed in cross section through a plane extending laterally through the microstructures and parallel to but offset from the substrate. The microstructures may include shape changes along a length of the microstructure. For example, microstructures could include a shoulder that is configured to abut against the stratum corneum to control a depth of penetration and/or a shaft extending to the tip, with the shaft being configured to control a position of the tip in the subject and/or provide a surface for an electrode.

Different microstructures could be provided on a common substrate, for example providing different shapes of microstructure to achieve different functions. In one example, this could include performing different types of measurement. In other examples, microstructures could be provided on different substrates, for example, allowing sensing to be performed via microstructures on one patch and delivery of therapy to be performed via microstructures on a different patch. In this example, this could allow a therapy patch to be replaced once exhausted, whilst a sensing patch could remain in situ. Additionally, measurements could be performed between patches, for example, performing whole of body, or body segment, such as limb, or part of body, impedance measurements between patches provided at different locations on a subject.

Additionally and/or alternatively anchor microstructures could be provided, which can be used to anchor the substrate to the subject. In this regard, anchor microstructures would typically have a greater length than that of the microstructures, which can help retain the substrate in position on the subject and ensure that the substrate does not move during the measurements or is not being inadvertently removed. Anchor microstructures can include anchoring structures, such as raised portions, which can assist with engaging the tissue, and these could be formed by a shape of the microstructure and/or a shape of a coating. Additionally, the coating could include a hydrogel or other similar material, which expands upon expose to moisture within the subject, thereby further facilitating engagement with the subject. Similarly the microstructure could undergo a shape change, such as swelling either in response to exposure to substances, such as water or moisture within the subject, or in response to an applied stimulation. When applied to skin, the anchor microstructures can enter the dermis, and hence are longer than other microstructures, to help retain the substrate in place, although it will be appreciated that this is not essential and will depend upon the preferred implementation. In other examples the anchor microstructures are rougher than other microstructures, have a higher surface friction than other microstructures, are blunter than other microstructures or are fatter than other microstructures.

In a further example, at least part of the substrate could be coated with an adhesive coating in order to allow the substrate and hence patch, to adhere to the subject.

As previously mentioned, when applied to skin, the microstructures typically enter the viable epidermis and in one example, do not enter the dermis, although in other examples, may enter the dermis. But this is not essential, and for some applications, it may be necessary for the microstructures to enter the dermis, for example projecting shortly through the viable epidermis/dermis boundary or entering into the dermis a significant distance, largely depending on the nature of the sensing being performed. In one example, for skin, the microstructures have a length that is at least one of less than 2500 μm, less than 1000 μm, less than 750 μm, less than 600 μm, less than 500 μm, less than 400 μm, less than 300 μm, less than 250 μm, greater than 100 μm, greater than 50 μm and greater than 10 μm, but it will be appreciated that other lengths could be used. More generally, when applied to a functional barrier, the microstructures typically have a length greater than the thickness of the functional barrier, at least 10% greater than the thickness of the functional barrier, at least 20% greater than the thickness of the functional barrier, at least 50% greater than the thickness of the functional barrier, at least 75% greater than the thickness of the functional barrier and at least 100% greater than the thickness of the functional barrier.

In another example, the microstructures have a length that is no more than 2000% greater than the thickness of the functional barrier, no more than 1000% greater than the thickness of the functional barrier, no more than 500% greater than the thickness of the functional barrier, no more than 100% greater than the thickness of the functional barrier, no more than 75% greater than the thickness of the functional barrier or no more than 50% greater than the thickness of the functional barrier. This can avoid deep penetration of underlying layers within the body, which can in turn be undesirable, and it will be appreciated that the length of the microstructures used will vary depending on the intended use, and in particular the nature of the barrier to be breached, and/or signals to be applied or measured. The length of the microstructures can also be uneven, for example, allowing a blade to be taller at one end than another, which can facilitate penetration of the subject or functional barrier.

Similarly, the microstructures can have different widths depending on the preferred implementation. Typically, the widths are at least one of less than 25% of the length, less than 20% of the length, less than 15% of the length, less than 10% of the length, or less than 5% of the length. Thus, for example, when applied to the skin, the microstructures could have a width of less than 50 μm, less than 40 μm, less than 30 μm, less than 20 μm or less than 10 μm. However, alternatively, the microstructures could include blades, and could be wider than the length of the microstructures. In some example, the microstructures could have a width of less than 50000 μm, less than 40000 μm, less than 30000 μm, less than 20000 μm, less than 10000 μm, less than 5000 μm, less than 2500 μm, less than 1000 μm, less than 500 μm or less than 100 μm. In blade examples, it is also feasible to use microstructures having a width substantially up to the width of the substrate.

In general the thickness of the microstructures is significantly lower in order to facilitate penetration and is typically less than 1000 μm, less than 500 μm, less than 200 μm, less than 100 μm, less than 50 μm, less than 20 μm, less than 10 μm, at least 1 μm, at least 0.5 μm or at least 0.1 μm. In general the thickness of the microstructure is governed by mechanical requirements, and in particular the need to ensure the microstructure does not break, fracture or deform upon penetration. However, this issue can be mitigated through the use of a coating that adds additional mechanical strength to the microstructures.

In one specific example, for epidermal sensing, the microstructures have a length that is less than 300 μm, greater than 50 μm, greater than 100 μm and about 150 μm, and, a width that is greater than or about equal to a length of the microstructure, and is typically less than 300 μm, greater than 50 μm and about 150 μm. In another example, for dermal sensing, the microstructures have a length that is less than 450 μm, greater than 100 μm, and about 250 μm, and, a width that is greater than or about equal to a length of the microstructure, and at least of a similar order of magnitude to the length, and is typically less than 450 μm, greater than 100 μm, and about 250 μm. In other examples, longer microstructures could be used, so for example for hyperdermal sensing, the microstructures would be of a greater length. The microstructures typically have a thickness that is less than the width, significantly less than the width and of an order of magnitude smaller than the width. In one example, the thickness is less than 50 μm, greater than 10 μm, and about 25 μm, whilst the microstructure typically includes a flared base for additional strength, and hence includes a base thickness proximate the substrate that is about three times the thickness, and typically is less than 150 μm, greater than 30 μm and about 75 μm. The microstructures typically have a tip with a length that is less than 50% of a length of the microstructure, at least 10% of a length of the microstructure and more typically about 30% of a length of the microstructure. The tip further has a sharpness that is at least 0.01 μm, at least 0.05 μm, at least 0.1 μm, less than 5 μm and typically about 1 μm.

In one example, the microstructures have a relatively low density, such as less than 10000 per cm², such as less than 1000 per cm², less than 500 per cm², less than 100 per cm², less than 10 per cm² or even less than 5 per cm². The use of a relatively low density facilitates penetration of the microstructures through the stratum corneum and in particular avoids the issues associated with penetration of the skin by high density arrays, which in turn can lead to the need for high powered actuators in order for the arrays to be correctly applied. However, this is not essential, and higher density microstructure arrangements could be used, including less than 50,000 microstructures per cm², less than 30,000 microstructures per cm², or the like. As a result, the microstructures typically have a spacing that is less than 20 mm, less than 10 mm, less than 1 mm, less than 0.1 mm or less than 10 μm. It should be noted that in some circumstances, microstructures are arranged in pairs, with the microstructures in each pair having a small spacing, such as less than 10 μm, whilst the pairs have a greater spacing, such as more than 1 mm, in order to ensure a low overall density is maintained. However, it will be appreciated that this is not essential, and higher densities could be used in some circumstances.

In one specific example, the microstructures have a density that is less than 5000 per cm², greater than 100 per cm², and about 25-50 per cm², or 600 per cm², depending on the intended applications. This leads to a spacing of less than 1 mm, more than 10 μm, and about 0.5 mm, 0.2 mm or 0.1 mm.

In one example, the microstructures include plates having a substantially planar face having an electrode thereon. The use of a plate shape maximizes the surface area of the electrode, whilst minimizing the cross sectional area of the microstructure, to thereby assist with penetration of the microstructure into the subject. This also allows the electrode to act as a capacitive plate, allowing capacitive sensing to be performed. In one example, the electrodes have a surface area of at least at least 10 mm², at least 1 mm², at least 100,000 μm², 10,000 μm², at least 7,500 μm², at least 5,000 μm², at least 2,000 μm², at least 1,000 μm², at least 500 μm², at least 100 μm², or at least 10 μm². In one example, the electrodes have a width or height that is up to 2500 μm, at least 500 μm, at least 200 μm, at least 100 μm, at least 75 μm, at least 50 μm, at least 20 μm, at least 10 μm or at least 1 μm. In the case of electrodes provided on blades, the electrode width could be less than 50000 μm, less than 40000 μm, less than 30000 μm, less than 20000 μm, less than 10000 μm, or less than 1000 μm, as well as including widths outlined previously. In this regard, it will be noted that these dimensions apply to individual electrodes, and in some examples each microstructure might include multiple electrodes.

In one specific example, the electrodes have a surface area of less than 0.2 mm², at least 0.01 mm² and at least one of about 0.13 mm², 0.07 mm² and 0.02 mm², depending on the configuration. The electrodes extending over a length of a distal portion of the microstructure, optionally spaced from the tip, and optionally positioned proximate a distal end of the microstructure, again proximate the tip of the microstructure. The electrode can extend over at least 25% and less than 50% of a length of the microstructure, so that the electrode typically extends over about 60 μm 90 μm or 150 μm of the microstructure and hence is positioned in a viable epidermis and/or dermis of the subject in use.

In one example, at least some of the microstructures are arranged in groups, such as pairs, with response signals or stimulation being measured from or applied to the microstructures within the group. The microstructures within the group can have a specific configuration to allow particular measurements to be performed. For example, when arranged in pairs, a separation distance can be used to influence the nature of measurements performed. For example, when performing bioimpedance measurements, if the separation between the microstructures is greater than a few millimetres, this will tend to the measure properties of interstitial fluid located between the electrodes, whereas if the distance between the microstructures is reduced, measurements will be more influenced by surface properties, such as the presence of materials bound to the surface of the microstructures. Measurements are also influenced by the nature of the applied stimulation, so that for example, current at low frequencies will tend to flow though extra-cellular fluids, whereas current at higher frequencies is more influenced by intra-cellular fluids.

In one particular example, plate microstructures are provided in pairs, with each pair including spaced apart plate microstructures defining substantially planar electrodes in opposition. This can be used to generate a highly uniform field in the subject in a region between the electrodes, and/or to perform capacitive or conductivity sensing of substances between the electrodes. This effect can be further enhanced by providing rows of microstructures, with microstructures within a row being electrically connected so that measurements can be performed between rows of microstructures, thereby increasing the effective electrode surface area. However, this is not essential, and other configurations, such as circumferentially spacing a plurality of electrodes around a central electrode, can be used. Typically the spacing between the electrodes in each group is less than 50 mm; less than 20 mm, less than 10 mm, less than 1 mm, less than 0.1 mm or less than 10 μm, although it will be appreciated that greater spacings could be used, including spacing up to dimensions of the substrate and/or greater, if microstructures are distributed across multiple substrates.

Thus, in one specific example, at least some of the microstructures are arranged in pairs or pairs of rows, with response signals being measured between microstructures in the pair or row pair and/or stimulation being applied between microstructures in the pair or row pair. Each pair of microstructures typically includes spaced apart plate microstructures having substantially planar electrodes in opposition and/or spaced apart substantially parallel plate microstructures.

In one example, at least some pairs of microstructures are angularly offset, and in one particular example, are orthogonally arranged. Thus, in the case of plate microstructures, at least some pairs of microstructures extend in different and optionally orthogonal directions. This distributes stresses associated with insertion of the patch in different directions, and also acts to reduce sideways slippage of the patch by ensuring plates at least partially face a direction of any lateral force. Reducing slippage either during or post insertion helps reduce discomfort, erythema, or the like, and can assist in making the patch comfortable to wear for prolonged periods. Additionally, this can also help to account for any electrical anisotropy within the tissue, for example as a result of fibrin structures within the skin, cellular anisotropy, or the like.

In one specific example, adjacent pairs of microstructures are angularly offset, and/or orthogonally arranged, and additionally and/or alternatively, pairs of microstructures can be arranged in rows, with the pairs of microstructures in one row are orthogonally arranged or angularly offset relative to pairs of microstructures in other rows.

In one specific example, when pairs of microstructures are used, a spacing between the microstructures in each pair is typically less than 0.25 mm, more than 10 μm and about 0.1 mm, whilst a spacing between groups of microstructures is typically less than 1 mm, more than 0.2 mm and about 0.5 mm. Such an arrangement helps ensure electrical signals are primarily applied and measured within a pair and reduces cross talk between pairs, allowing independent measurements to be recorded for each pair of microstructures/electrodes.

To create an array of pairs of electrodes, this can be performed by manufacturing a first substrate having first microstructures and corresponding first apertures. An insulating layer is then provided on a side of the first substrate opposite the first microstructures before a second substrate is provided on the insulating layer. In this example, the second substrate has second microstructures extending through the insulating layer and the first apertures to form pairs of first and second microstructures, and an example of this will be described in more detail below. In one example, the first and second apertures are offset to reduce capacitive coupling between the first and second substrates. Alternatively, other mechanisms for capacitive coupling between the substrates could be used.

The microstructures can be configured in order to interact with, and in particular, bind with one or more analytes of interest, allowing these to be detected. Specifically, in one example, binding of one or more analytes to the microstructures can alter the charge carrying capability, in turn leading to changes in capacitance of electrode pairs, which can then be monitored, allowing analyte levels or concentrations to be derived. Binding of analytes can be achieved using a variety of techniques, including selection of mechanical properties of the microstructure, such as the presence of pores or other physical structures, the material from which the microstructures are manufactured, the use of coatings, or otherwise influencing the microstructure properties, such as by using magnetic microstructures.

Additionally, the microstructures and/or substrate can incorporate one or more materials or other additives, either within the body of the microstructure, or through addition of a coating containing the additive. The nature of the material or additive will vary depending on the preferred implementation and could include a bioactive material, a reagent for reacting with analytes in the subject, a binding agent for binding with analytes of interest, a material for binding one or more analytes of interest, a probe for selectively targeting analytes of interest, a material to reduce biofouling, a material to attract at least one substance to the microstructures, a material to repel or exclude at least one substance from the microstructures, a material to attract at least some analytes to the microstructures, or a material to repel or exclude analytes. In this regard, substances could include any one or more of cells, fluids, analytes, or the like. Example materials include polyethylene, polyethylene glycol, polyethylene oxide, zwitterions, peptides, hydrogels and self-assembled monolayers.

The material can be contained within the microstructures themselves, for example by impregnating the microstructures during manufacture, for example by introducing the material into the imprinting mold so that the material is applied to the microstructure surface during the molding process. Additionally and/or alternatively, the material can be incorporated into the formable material prior to molding, or could be provided in a coating.

Accordingly, it will be appreciated that at least some of the microstructures can include a material for binding one or more analytes or interest, which can be used in order to target specific analytes of interest, allowing these to bind or otherwise attach to the microstructure, so that these can then be detected in situ using a suitable detection mechanism, such as by detecting changes in optical or electrical properties.

The analyte may be any compound able to be detected in the epidermis and/or dermis. In particular embodiments, the analyte is a marker of a condition, disease, disorder or a normal or pathologic process that occurs in a subject, or a compound which can be used to monitor levels of an administered substance in the subject, such as a medicament (e.g., drug, vaccine), an illicit substance (e.g. illicit drug), a non-illicit substance of abuse (e.g. alcohol or prescription drug taken for non-medical reasons), a poison or toxin, a chemical warfare agent (e.g. nerve agent, and the like) or a metabolite thereof. Suitable analytes include, but are not limited to a:
nucleic acid, including DNA and RNA, including short RNA species including microRNA, siRNA, snRNA, shRNA and the like;
antibody, or antigen-binding fragment thereof, allergen, antigen or adjuvant;
chemokine or cytokine;
hormone;
parasite, bacteria, virus, or virus-like particle, or a compound therefrom, such as a surface protein, an endotoxin, and the like;
epigenetic marker, such as the methylation state of DNA, or a chromatin modification of a specific gene/region;
peptide;
polysaccharide (glycan);
polypeptide;
protein; and
small molecule.

In particular embodiments, the analyte of interest is selected from the group consisting of a nucleic acid, antibody, peptide, polypeptide, protein and small molecule; especially a polypeptide and protein; most especially a protein.

In a further example, one or more microstructures include a treatment material, and wherein at least one treatment delivery mechanism is provided that controls release of the treatment material. In one preferred example, release of the treatment material is controlled by applying stimulation to the microstructure(s), for example by applying light, heat or electrical stimulation to release the treatment material.

In one preferred example, the treatment material is contained in a coating on the at least one microstructure and the stimulation is used to dissolve the coating on the microstructure and thereby deliver the treatment material. It will be appreciated that this technique can be applied to any treatment material that can be incorporated into a coating, and which can be selectively released using stimulation, such as mechanical, magnetic, thermal, electrical, electromagnetic or optical stimulation.

The nature of the treatment material will vary depending on the preferred implementation and/or the nature of the treatment being performed, including whether the treatment is cosmetic or therapeutic. Example treatment materials include, but are not limited to, nanoparticles, a nucleic acid, an antigen or allergen, parasites, bacteria, viruses, or virus-like particles, metals or metallic compounds, molecules, elements or compounds, DNA, protein, RNA, siRNA, sfRNA, iRNA, synthetic biological materials, polymers, drugs, or the like.

It will be appreciated that the use of coatings is not essential however, and additionally and/or alternatively treatment materials can be incorporated into the microstructures themselves.

Irrespective of how treatment materials are provided, the substrate can include a plurality of microstructures with different microstructures having different treatment materials and/or different treatment doses. In this case, the processing devices can control the therapy delivery mechanism to release treatment material from selected microstructures, thereby allowing different treatments to be administered, and/or allowing differential dosing, depending on the results of measurements performed on the subject. In particular, as will be described in more detail below, the processing devices typically perform an analysis at least in part using the measured response signals; and, use results of the analysis to control the at least one therapy delivery mechanism, thereby allowing personalised treatment to be administered substantially in real time.

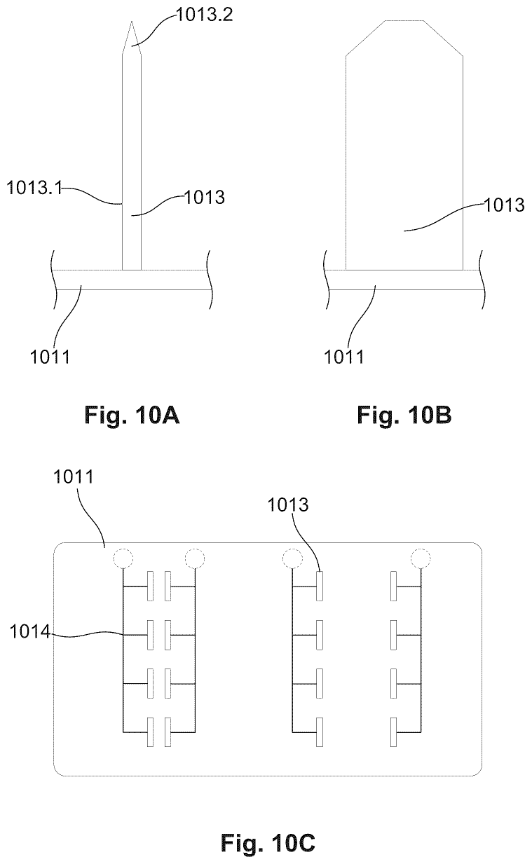

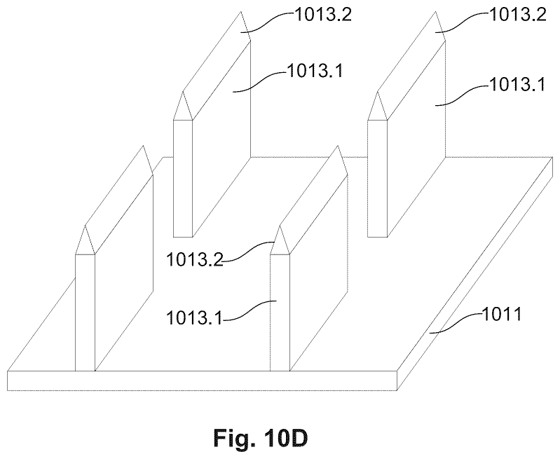

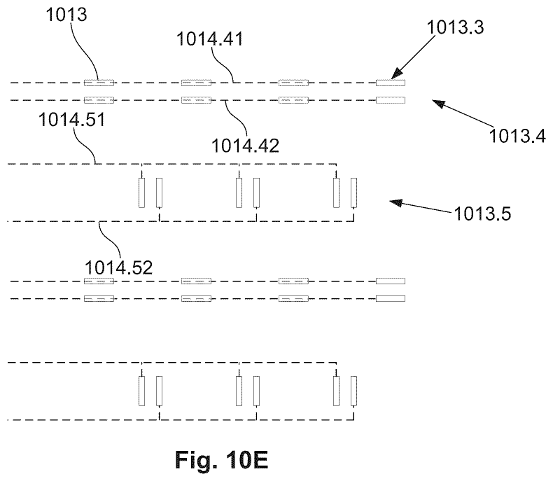

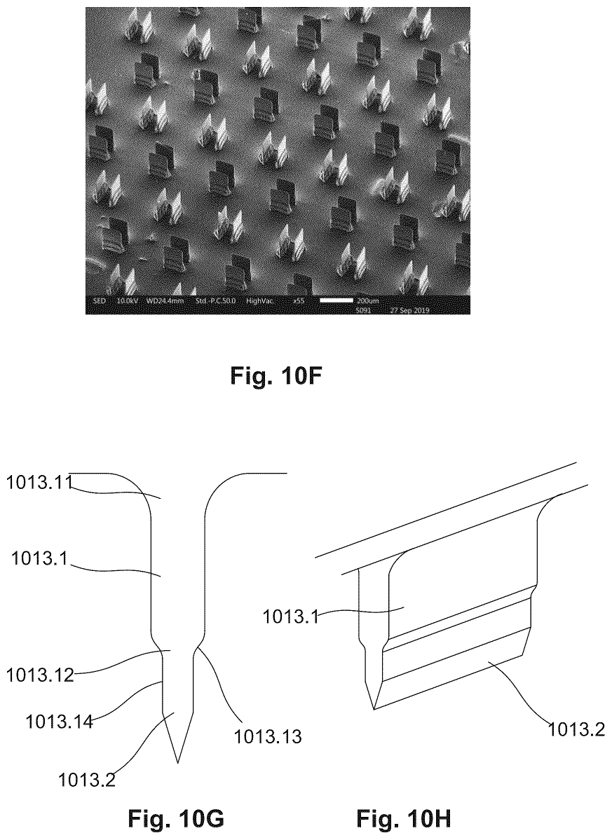

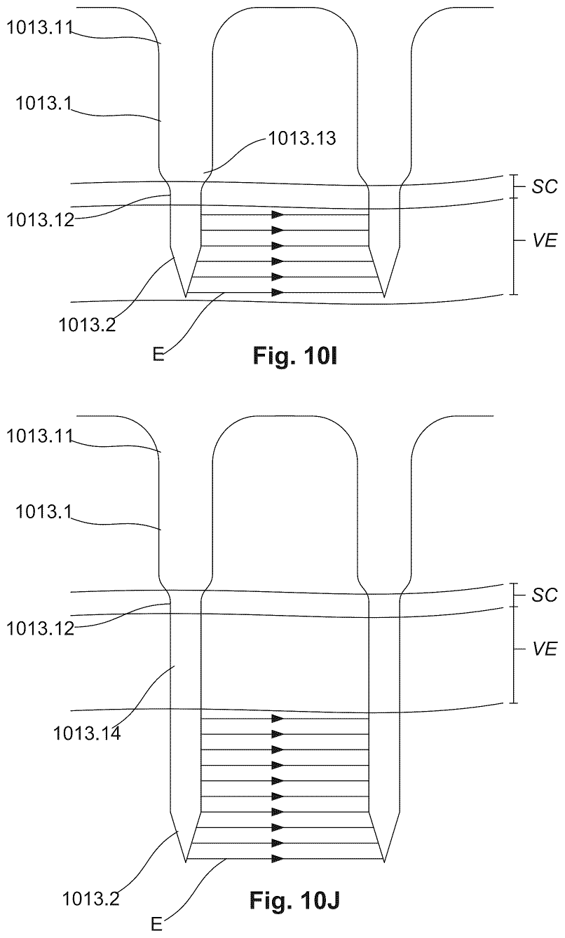

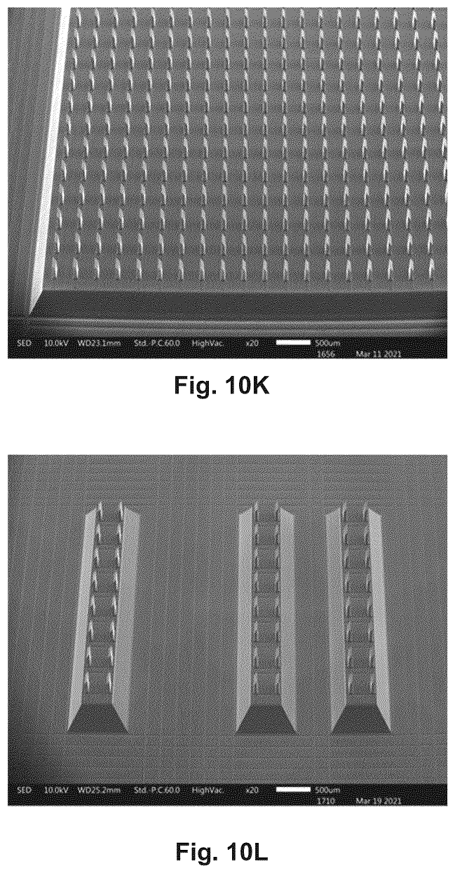

A specific example of a plate microstructure is shown is shown in FIGS. 10A to 10D.

In this example, the microstructure is a plate having a body 1013.1 extending from the substrate 1011, and a tip 1013.2, which is tapered to facilitate penetration of the microstructure 1013 into the stratum corneum. As shown in FIGS. 10C and 10D, different arrangements could be used but in general, pairs of microstructures are formed with the microstructures facing each other allowing signals to be applied between the microstructures or measured between the microstructures. Different separations between electrodes in pairs of electrodes can be used to allow different measurements to be performed and/or to alter the profile of stimulation of the tissue between the electrodes.

In the example of FIG. 10C, the pairs of microstructures are arranged in rows, with electrical connections 1014 extending to each microstructure in the row. It will be appreciated that in practice this can be achieved by ensuring the microstructures in each row are electrically connected, with openings being provided between the rows to electrically isolate the rows.

A further example arrangement is shown in FIG. 10E, in which microstructures 1013 are arranged in pairs 1013.3, and with pairs arranged in offset rows, 1013.4, 1013.5. In this example, pairs in different rows are arranged orthogonally, so that the microstructures extend in different directions. This avoids all microstructures being aligned, which can in turn render a patch vulnerable to lateral slippage in a direction aligned with the microstructures. Additionally arranging the pairs orthogonally reduces interference, such as cross talk, between different pairs of electrodes, improving measurement accuracy and accounting for tissue anisotropy, particularly when measurements are being performed via multiple microstructure pairs simultaneously.

In one example, pairs of microstructures in each row can be provided with respective connections 1014.41, 1014.42; 1014.51, 1014.52, allowing an entire row of microstructure pairs to be interrogated and/or stimulated simultaneously, whilst allowing different rows to be interrogated and/or stimulated independently.

A Scanning Electron Microscopy (SEM) image showing an array of pairs of offset plate microstructures is shown in FIG. 10F.

Specific examples of microstructures for performing measurements in the epidermis are shown in FIGS. 10G and 10H.

In this example, the microstructures are plates or blades, having a body 1013.1, with a flared base 1013.11, where the body joins the substrate, to enhance the strength of the microstructure. The body narrows at a waist 1013.12 to define shoulders 1013.13 and then extends to a tapered tip 1013.2, in this example, via an untapered shaft 1013.14. Typical dimensions are shown in Table 2 below.

TABLE 2

| Parameter | Min. | Typical | Max. | Units |
|---|---|---|---|---|
| Length | 50 | 150 | 300 | microns |
| Width | 50 | 150 | 300 | microns |
| Thickness | 10 | 25 | 50 | microns |
| Density | 100 | 600 | 5000 | cm⁻² |
| Tip radius | 0.1 | >1 | 5 | microns |
| Surface area per electrode | 2,000 | 22,500 | 200,000 | micron² |
| Buttress width at base | 30 | 75 | 150 | microns |

An example of a pair of the microstructures of on insertion into a subject is shown in FIG. 10I.

In this example, the microstructures are configured so that the tip 1013.2 penetrates the stratum corneum SC and enters the viable epidermis VE. The waist 1013.12, and in particular the shoulders 1013.13 abut the stratum corneum SC so that the microstructure does not penetrate further into the subject, and so that the tip is prevented from entering the dermis. This helps avoid contact with nerves, which can lead to pain.

In this configuration, the body 1013.1 of the microstructure can be coated with a layer of insulating material (not shown), with only the tip exposed. As a result a current signal applied between the microstructures, will generate an electric field E within the subject, and in particular within the viable epidermis VE, so that measurements reflect fluid levels in the viable epidermis VE.

However, it will be appreciated that other configurations can be used. For example, in the arrangement of FIG. 10J, the shaft 1013.14 is lengthened so the tip 1013.2 enters the dermis, allowing dermal (and optional epidermal) measurements to be performed.

In this example, typical dimensions are shown in Table 3 below.

TABLE 3

| Parameter | Min. | Typical | Max. | Units |
|---|---|---|---|---|
| Length | 50 | 250 | 450 | microns |
| Width | 50 | 250 | 450 | microns |
| Thickness | 10 | 30 | 50 | microns |
| Density | 100 | 600 | 5000 | cm⁻² |
| Tip radius | 0.1 | 1 | 5 | microns |
| Surface area per electrode | 10,000 | 62,500 | 427,000 | micron² |
| Buttress width at base | 30 | 75 | 150 | microns |

An example of the inter and intra pair spacing for these configurations are shown in Table 4 below.

TABLE 4

| Parameter | Min. | Typical | Max. | Units |
|---|---|---|---|---|
| Separation between | 10 | 100 | 1000 | microns |
| microstructures | | | | |
| in a group or pair | | | | |
| Separation between | 200 | 1000 | 5000 | microns |
| groups of | | | | |
| microstructures | | | | |

As mentioned above, the molding process uses a mold having microstructure-shaped cavities. To form the mold, a master patch is created, typically using silicon etching or a similar process, to create a master that is highly accurate and durable.

Images of further example microstructures manufactured using the above described techniques are shown in FIGS. 10K and 10L.

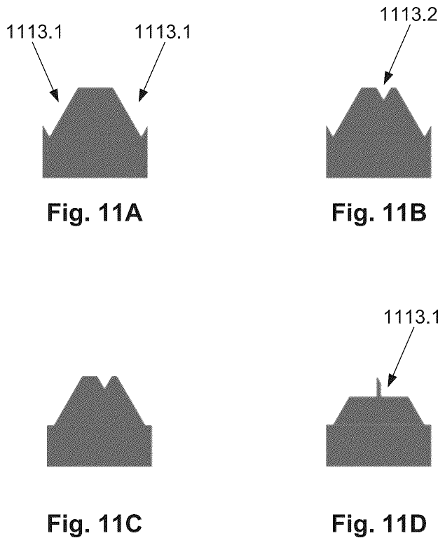

An example of a sequence of steps to etch a master microstructure for the mesa arrangement of FIG. 9C will now be described with reference to FIGS. 11A to 11D.

Initially, the silicon is etched to create mesa bevelling 1113.1 as shown in FIG. 11A, with a needle tip profile 1113.2 being etched as shown in FIG. 11B. The mesa is then cleared as shown in FIG. 11C, before the top of the mesa is removed to create the microstructure body 1113.1 as shown in FIG. 11D.

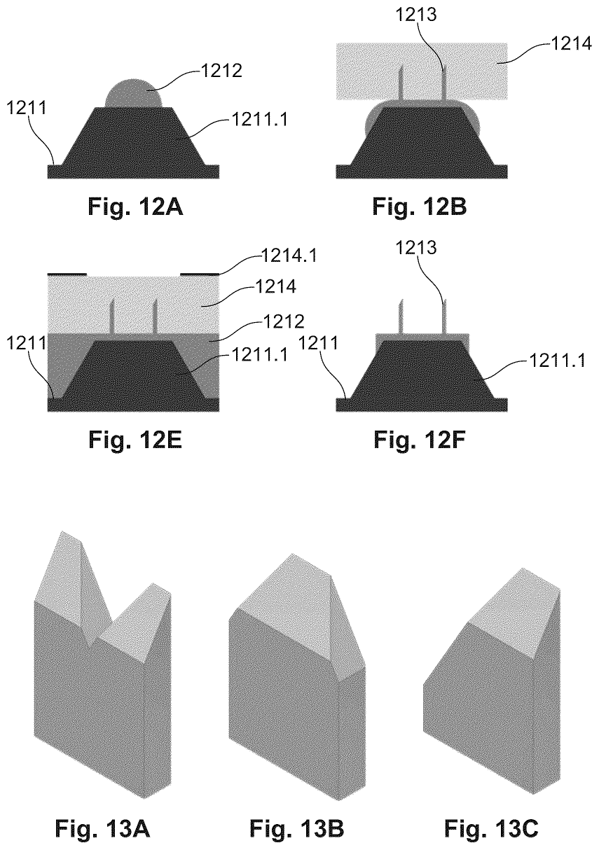

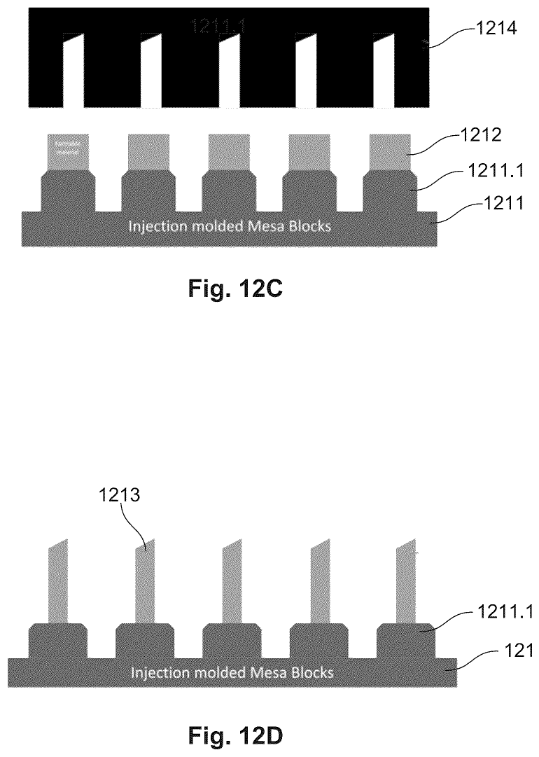

The above examples have focused on using a substantially planar substrate, but this is not essential and pre-patterned substrates with surface relief or features can be used. An example of this is shown in FIGS. 12A and 12B, and further in FIGS. 12C and 12D, where a substrate 1211 is pre-patterned with mesas 1211.1. In this example, the formable material 1212 is applied to the mesa top and the mold 1214 used to create the microstructures 1213. Thus, this example provides an alternate method for manufacturing microstructures which combines the manufacturing of support substrates with mesa platforms and the imprinting of formable material selectively deposited on top of the mesa blocks. This approach allows for the patches to be manufactured cheaply and at high volumes, making the resulting devices readily available. It further enables generating patches at low mix/high volume or high mix/high volume as the imprint happens only on those regions where the formable material is present.

In one example, the injection molding of the mesa blocks requires a micro-insert carrying the inverse polarity of the mesas that need to be manufactured. Usually, the micro-inserts are made of stainless, Ni or metal alloys. Such method of manufacturing enables thousands of mesa block shots to be manufactured within a brief period, say, in few hours.

The selective positioning of the formable material 1212 can be performed using automated dispensers, ink jet coating that are guided with pre-set geometrical values to precisely coat the selective areas as required. An appropriate coating material or primer can be applied on mesa blocks so that the formable material adheres well after imprinting.

The examples for injection molding polymer include cyclo olefin polymer, polyvinyl chloride, polypropylene, polyethylene terephthalate, poly methyl methacrylate, high density polyethylene, low density polyethylene, Acrylonitrile Butadiene Styrene, Styrene, Styrene Acrylonitrile, polyamides, Polyoxymethylene etc.

The imprint mold carries features with inverse polarity (vias) which when used to imprint the formable material will result in microstructures, such as blades. The advantage is that, although the mold may carry features on its entire surface, the microstructure imprints will be produced only on those areas where the formable material is selectively positioned on the injection molded substrates carrying mesa blocks. The design of the mold and imprint process can be tuned such that each mesa block has one microblade (one-to-one) or multiple blades (one-to-many).

It will be appreciated that a wide range of different surface features could be incorporated into the substrate, including but not limited to vias, mesas, channels, or the like.

In another example, curing can be facilitated through the use of a shadow mask incorporated into the mold, and an example of this is shown in FIGS. 12E and 12F.

In this example, a formable material 1212 is applied to the pre-patterned substrate 1211 of FIG. 12A, coating the mesa and the surrounding substrate surface. The mold 1214 which is applied includes a mask 1214.1, which is aligned with the area surrounding the mesa, so that the formable material in region of the mesa can be exposed and hardened, allowing the remaining formable material to be removed as shown in FIG. 12D. It will be appreciated that although this example was described using a pre-patterned substrate this is not essential and the approach could also be used on non-patterned substrates.

It will be noted that whilst the description and drawings have focused on the creation of plate or blade style microstructures, this is not essential and a wide range of different shaped microstructures could be manufactured using these techniques, including microneedles, and other shapes, and further including microstructures including channels to allow for sampling of fluid from within the subject.

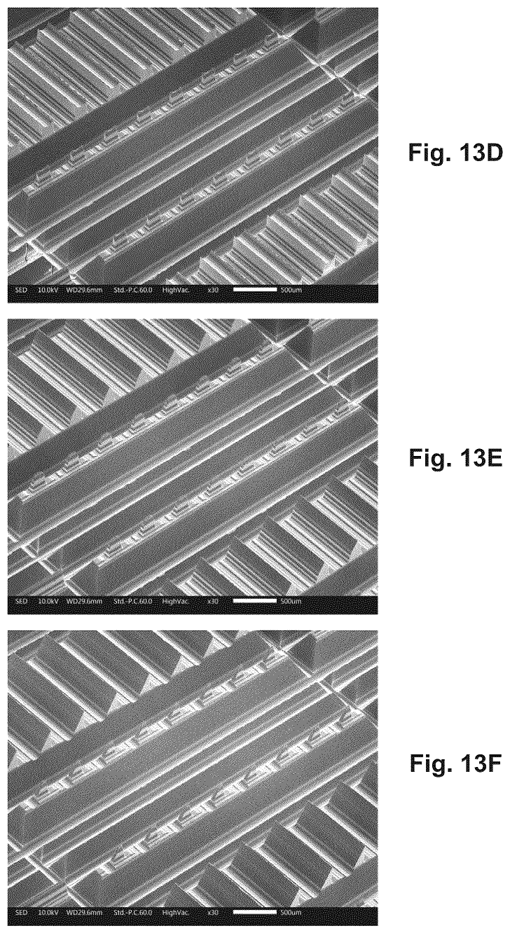

Additionally, complex microstructure geometries can be easily manufactured using these techniques. Examples of complex master projections manufactured using wafer slicing are shown in FIGS. 13A to 13C, with corresponding microstructures manufactured using these approaches being shown in FIGS. 13D to 13F.

It will therefore be appreciated that throughout the above, use of blade or plate type microstructures is for the purpose of illustration only and is not intended to be limiting.

A further specific example of polymer microstructures created using the above described approach, and specifically using nano imprint lithography, will now be described.

In this example, silicon master molds with pre-defined microprojections were used to create an intermediate polymer stamp (IPS) which mirrored the master molds surface topology. The latter was in turn used to imprint resins to create microprojections with geometry similar to the master mold.

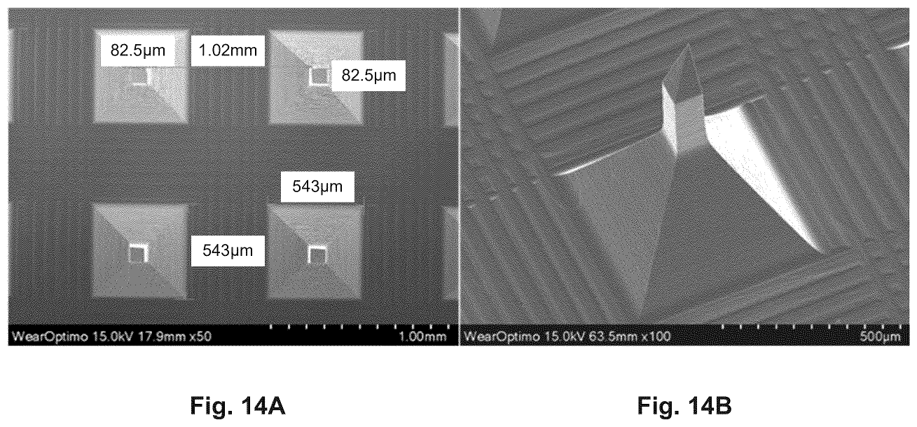

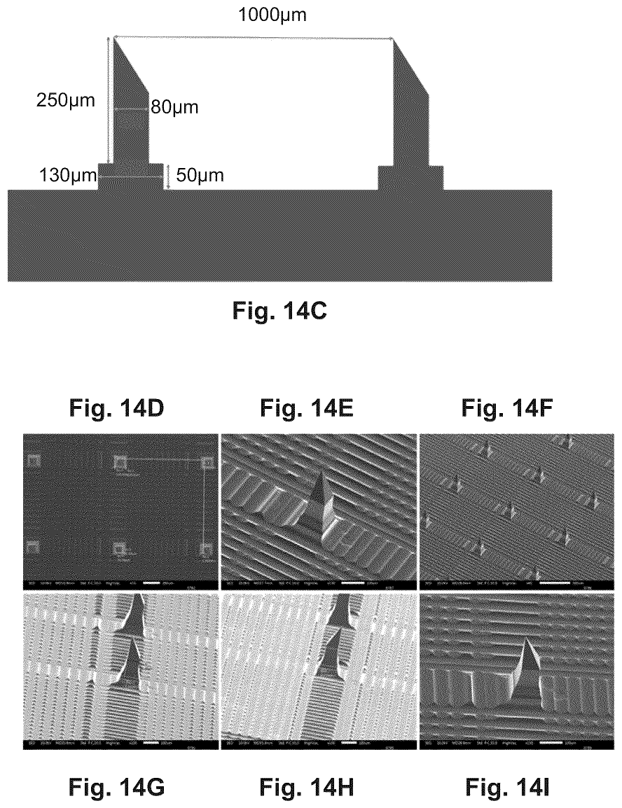

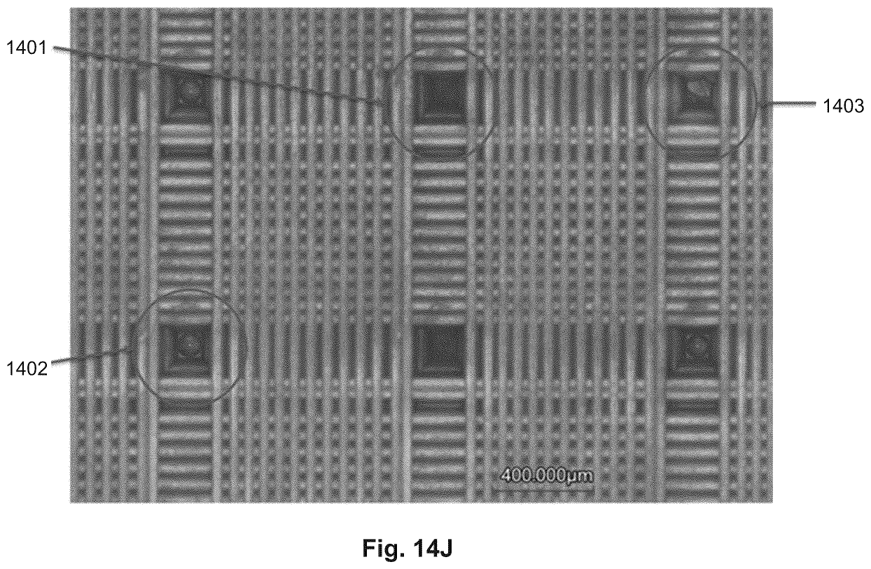

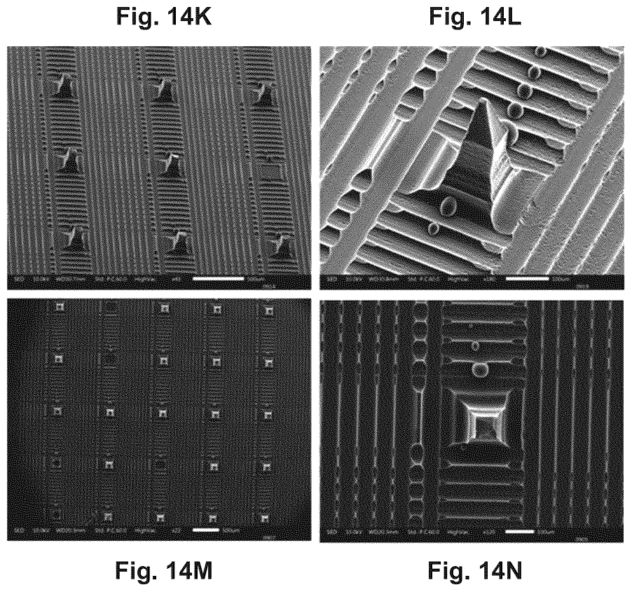

A 6″ Si mold (M1), with the thickness of 1300 μm was used to fabricate the master mold shown in FIGS. 14A and 14B. Together with the mesas at 450 μm and projections at 350 μm, the features reached a total height of ~800 μm. After dicing, a 500 μm of the undiced silicon remained. While using thick silicon, a significant residual Si was left behind after the microfeatures were carved from the dicing process. This ensured a robust bottom support needed during during the fabrication of the IPS, which is then used for imprinting. The top view of the substrate shown in FIG. 14A displays the size of the mesa to be around 550 μm×550 μm and microneedles ~80 μm×80 μm, spaced 1 mm apart. The wafer was patterned with 5×5 arrays of microprojections of size 5.5 mm×5.5 mm.

While developing process for imprinting, the aspect ratio used in M1 turned out to be too high for any reasonable IPS to be fabricated and therefore, the features heights were modified while retaining the same feature morphology, as shown in FIG. 14C. The total height was reduced to ~320 μm (~260 μm microneedles, ~60 μm mesa). Cubic mesas, instead of pyramids were fabricated and with the reduction in the feature heights, a thinner wafer (1 mm) for both the masters proved sufficient. The new master mold (M2) of the same morphology is shown in was fabricated on square wafers of 10.6 cm×10.6 cm with 3 cm bleeding area around the active area. The distance between micro projections and chips size remained consistent with the first wafer. SEM images of the second master mold are shown in FIGS. 14D to 14I.

M2 was then used to prepare the intermediate polymer stamp (IPS) typically made from polymers with low surface energy, which typically contain fluoropolymers. In one example, the IPS uses FlexStamp from Morphotonics™. Other examples of IPS include Polydimethylsiloxane (PDMS) from Dow Corning, OrmoStamp from microresist technology, or the like. The IPS was then used for the trials in imprinting different resins that would allow replication of the features as close as possible to the Si features. While the IPS was fabricated, the quality of the replication could be checked only by making an imprint. Imprints were made using resin MM1158. The resin also exhibited good wetting behavior. The imprint conditions used were-Speed: 50 mm/min; Pressure 10 N/cm.

An optical microscopy image of the resulting structures is shown in FIG. 14J. The imprint showed some broken needles 1403 arising from the cured resin that stuck within the vias of the IPS while demolding, and some 1402 that arose from trapping of air bubbles arising from capillary action. Some fully formed needles 1401 were also observed.

During process development it was observed that the change in aspect ratio allowed enhanced filling of the resins within the IPS features. Using this as the basis, further optimization was carried out and better results were obtained under the following conditions-speed 50 mm/min, pressure 10 N/cm during the first two laminations (without curing) and 7 N/cm during the third lamination (curing step). This resulted in slightly taller polymer needles and lesser air bubbles/traps. However, the needles still had broken features, incomplete filling but the results were better than the first trial.

A thorough study of the imprint was performed using SEM, and resulting images are shown in FIGS. 14K to 14N. As observed, the optimization process yielded several good microfeatures over a reasonable area. The tips of the microstructures were slightly rounded which can be attributed to the improper filling due to capillary action, air trapping inside the vias of the IPS features. The mesa was replicated almost completely. The microprojection shaft also showed good replication. Due to demolding, the sides of the imprinted feature were rough presumably due to some amount of resin delaminating together with the IPS. The cause of some missing features can be either due to vias being filled with air not allowing the resin to fill into or as the IPS was used several times, it could have been soiled with resin (broken needles within). A slight variation in the MM1158 with even lower surface free energy (SFE), seems to be good, however, this needs to be tested further.

Several resins were tested during this study and are captured in the table below,

| | Viscosity | Result for the | |
|---|---|---|---|
| Resin | (mPas, at 25° C.) | microneedles | Remark |
| MM1043C | 90 | Not good | Air inclusion |
| MM2138B2 | 35 | Not good | Air inclusion |
| MM2017A | 7 | Not good | Too low viscosity |
| MM2394G | 25 | Not good | Too low viscosity |
| MM1158 | 35 | Improvement | Low SFE |
| MM1158A | 35 | Improvement | Even lower SFE |

These results demonstrate the ability of imprinting techniques to generate usable microstructures.

Persons skilled in the art will appreciate that numerous variations and modifications will become apparent. All such variations and modifications which become apparent to persons skilled in the art, should be considered to fall within the spirit and scope that the invention broadly appearing before described.

The claims defining the invention are as follows:

1. A method for manufacturing a patch for application to a biological subject, the method including shaping a formable material to form a plurality of microstructures on a substrate, wherein the microstructures are shaped to breach a functional barrier of the subject.

2. A method according to claim 1, wherein the method includes:
a) providing a substrate;
b) depositing a formable material layer on the substrate;
c) forming a plurality of microstructures in the formable material layer, wherein the microstructures are shaped to breach a functional barrier of the subject; and,
d) solidifying the formable material.

3. A method according to claim 2, wherein the method includes forming the plurality of microstructures by one of:
a) hot embossing; and,
b) imprinting.

4. A method according to claim 2 or claim 3, wherein the formable material layer is imprinted using at least one of:
a) nano-imprint lithography;
b) roll to plate lithography;
c) roll to roll lithography;
d) plate to plate lithography; and,
e) plate to roll lithography.

5. A method for according to claim 1, wherein the method includes micro-injection molding a plurality of microstructures shaped to breach a functional barrier of the subject.

6. A method according to any one of the claims 1 to 5, wherein the formable material includes at least one of:
a) monomers;
b) oligomers;
c) photo-initiators;
d) crosslinking acrylate groups;
e) curable sol-gels;
f) epoxies;
g) resins;
h) polymers;
i) a curable formable material;
j) a UV curable formable material;
k) a sealant;
l) a UV glue
m) UV nano-imprint lithography polymers;
n) Helioseal;
o) urethane dimethacrylate;
p) bisphenol A-glycidyl methacrylate;
q) triethylene glycol dimethacrylate;
r) NOA 61;
s) Mercapto-ester*;
t) Triallyl Isocyanurate;
u) Inoflex RP+;
v) PAK 01;
w) NIF 2 (Asahi Glass Company);
x) NIF 1 (Asahi Glass Company); and,
y) Z Resist (t-butyl acrylate (96.5%), photoinitiator Irgacure 369 (3.5%));

7. A method according to claim 6, wherein the method includes solidifying the formable material using at least one of:
a) heat;
b) electromagnetic radiation;
c) visible electromagnetic radiation;
d) ultra-violet electromagnetic radiation; and,
e) by curing the material.

8. A method according to claim 6 or claim 7, wherein the formable material has at least one of:
a) a low viscosity;
b) a viscosity of at least one of:
c) between 10⁻³ Pa·s and 1 Pa·s; and,
d) between of 0.4-0.05 Pa·s.

9. A method according to any one of the claims 6 to 8, wherein the cured formable material has at least one of:
a) a high mechanical strength;
b) a high resistivity; and,
c) a high conductivity.

10. A method according to any one of the claims 1 to 9, wherein the substrate is at least one of:
a) one of:
i) substantially rigid; and,
ii) flexible;
b) polycarbonate;
c) polymethacrylimide (PMI);
d) polyethylene terephthalate (PET);
e) polycarbonate (PC);
f) poly(methyl methacrylate) (PMMA);
g) stainless steel;
h) metal;
i) polymer; and,
j) silicon.

11. A method according to any one of the claims 1 to 10, wherein the method includes at least one of:
a) applying at least one coating to at least part of at least some of the microstructures;
b) applying a primer prior to applying at least one coating; and,
c) applying a coating to formable material between at least some of the microstructures.

12. A method according to claim 11, wherein the coating includes at least one of:
a) a conductive coating;
b) a dielectric coating;
c) a mechanical coating;
d) gold;
e) titanium
f) titanium nitride;
g) metal;
h) ceramic;
i) parylene;
j) Poly(3,4-ethylene dioxythiophene); and
k) Iridium oxide (IrO₂);

13. A method according to claim 11 or claim 12, wherein the coating has a thickness of at least one of:
a) at least 10 nm;
b) at least 100 nm;
c) at least 200 nm;
d) at least 300 nm;
e) at least 400 nm;
f) at least 500 nm;
g) about 3 μm;
h) less than 4 μm; and,
i) less than 5 μm;

14. A method according to any one of the claims 11 to 13, wherein the method includes applying a coating using at least one of:
a) sputtering;
b) electroplating;
c) electroless plating;
d) spin coating;
e) thermal evaporation;
f) vapour deposition;
g) chemical vapour deposition;
h) plasma enhanced chemical vapour deposition; and,
i) ink-jet printing.

15. A method according to any one of the claims 1 to 14, wherein at least some of the microstructures are conductive microstructures and wherein the method includes creating the conductive microstructures by at least one of:
a) applying a conductive coating to at least some of the microstructures; and,
b) using a conductive formable material.

16. A method according to any one of the claims 1 to 15, wherein the method includes applying a dielectric coating to at least part of conductive microstructures.

17. A method according to claim 16, wherein the method includes:
a) applying a dielectric coating over conductive microstructures; and,
b) removing at least part of the dielectric coating to expose a surface of the conductive microstructure.

18. A method according to claim 16 or claim 17, wherein the microstructures include an dielectric coating extending over at least one of:
a) part of a surface of the microstructure;
b) a proximal end of the microstructure;
c) at least half of a length of the microstructure;
d) about 90 μm of a proximal end of the microstructure; and,
e) at least part of a tip portion of the microstructure.

19. A method according to any one of the claims 1 to 18, wherein at least some of the microstructures include at least one electrode that at least one of:
a) extends over a length of a distal portion of the microstructure;
b) extends over a length of a portion of the microstructure spaced from the tip;
c) is positioned proximate a distal end of the microstructure;
d) is positioned proximate a tip of the microstructure;
e) extends over at least 25% of a length of the microstructure;
f) extends over less than 50% of a length of the microstructure;
g) extends over about 60 μm of the microstructure; and,
h) is configured to be positioned in a viable epidermis of the subject in use.

20. A method according to any one of the claims 1 to 19, wherein the method includes one of electrically isolating at least some of the microstructures by at least one of:
a) removing material between the microstructures;
b) selectively coating at least some of the microstructures; and,
c) removing electrically conductive material between conductive microstructures to electrically isolate at least some of the microstructures.

21. A method according to claim 20, wherein the method includes removing material using at least one of:
a) etching;
b) laser ablation;
c) plasma etching;
d) chemical plasma etching;
e) inductively coupled plasma etching;
f) deep reactive ion etching;
g) physical etching;
h) oxygen plasma etching;
i) reactive-ion etching; and,
j) imprinting.

22. A method according to any one of the claims 1 to 21, wherein the formable material includes a lower layer adjacent the substrate and an upper layer incorporating the microstructures.

23. A method according to claim 22, wherein the method includes removing material by imprinting the upper layer.

24. A method according to any one of the claims 1 to 23, wherein the method includes providing electrical connections in electrical contact with at least some of conductive microstructures.

25. A method according to claim 24, wherein the electrical connections at least one of:
a) extend through vias in the substrate;
b) are provided on a surface of the substrate; and,
c) are provided on a surface of the formable material.

26. A method according to claim 24 or claim 25, wherein the formable material includes mesas, with the microstructures extending from the mesas, and with electrical connections provided between the mesas.

27. A method according to any one of the claims 1 to 26, wherein the method includes creating microfluidic channels in a surface of at least one of:
a) the substrate; and,
b) the formable material.

28. A method according to any one of the claims 1 to 27, wherein the method includes creating pores in at least some of the microstructures.

29. A method according to any one of the claims 1 to 28, wherein at least some of the microstructures are at least one of:
a) blades;
b) ridges;
c) needles; and,
d) plates.

30. A method according to any one of the claims 1 to 29, wherein at least some of the microstructures at least one of:
a) are at least partially tapered;
b) have a cross sectional shape that is at least one of:
i) circular;
ii) rectangular;
iii) cruciform;
iv) square;
v) rounded square;
vi) rounded rectangular;
vii) ellipsoidal; and,
viii) at least partially hollow;
c) have a surface that is at least partially at least one of:
i) smooth;
ii) serrated;
iii) includes one or more pores;
iv) includes one or more raised portions; and,
v) rough;
d) are at least partially hollow;
e) are porous; and,
f) include an internal structure.

31. A method according to any one of the claims 1 to 28, wherein the microstructures include at least one of:
a) plate microstructures;
b) at least partially tapered plate microstructures;
c) plate microstructures having a substantially rounded rectangular cross sectional shape;
d) spaced apart substantially parallel plate microstructures;
e) spaced apart rows of microstructures;
f) pairs of spaced apart microstructures; and,
g) groups of microstructures.

32. A method according to claim 31, wherein at least one of:
a) at least some microstructures are angularly offset;
b) at least some microstructures are orthogonally arranged;
c) adjacent pairs of microstructures are orthogonally arranged;
d) adjacent pairs of microstructures are angularly offset;
e) pairs of microstructures are arranged in rows, and the pairs of microstructures in one row are orthogonally arranged relative to pairs of microstructures in other rows; and,
f) pairs of microstructures are arranged in rows, and the pairs of microstructures in one row are angularly offset relative to pairs of microstructures in other rows.

33. A method according to any one of the claims 1 to 32, wherein the microstructures have a spacing that is at least one of:
a) less than 1 mm;
b) about 0.5 mm;
c) about 0.2 mm;
d) about 0.1 mm; and,
e) more than 10 μm.

34. A method according to any one of the claims 1 to 33, wherein at least some of the microstructures have at least one of:
a) a length that is at least one of:
i) less than 300 μm;
ii) about 150 μm;
iii) greater than 100 μm; and,
iv) greater than 50 μm;
b) a maximum width that is at least one of:
i) greater than the length;
ii) about the same as the length;
iii) less than 300 μm;
iv) about 150 μm; and,
v) greater than 50 μm; and,
c) a thickness that is at least one of:
i) less than 50 μm;
ii) about 25 μm; and,
iii) greater than 10 μm.

35. A method according to any one of the claims 1 to 34, wherein at least some of the microstructures have a tip that at least one of:
a) has a length that is at least one of:
i) less than 50% of a length of the microstructure;
ii) at least 10% of a length of the microstructure; and,
iii) about 30% of a length of the microstructure; and,
b) has a sharpness of at least one of:
i) at least 0.01 μm;
ii) at least 0.05 μm;
iii) at least 0.1 μm;
iv) less than 5 μm; and,
v) about 1 μm.

36. A method according to any one of the claims 1 to 35, wherein the microstructures have a density that is at least one of:
a) less than 5000 per cm²;
b) greater than 10 per cm²;
c) greater than 100 per cm²;
d) about 25-50 per cm²; and,
e) about 600 per cm².

37. A method according to any one of the claims 1 to 36, wherein at least some microstructures include an electrode having a surface area of at least one of:
a) less than 0.2 mm²;
b) about one of 0.13 mm², 0.07 mm² and 0.02 mm²; and,
c) at least 0.01 mm².

38. A method according to any one of the claims 1 to 37, wherein the microstructures include anchor microstructures used to anchor the substrate to the subject and wherein the anchor microstructures at least one of:
a) include anchoring structures;
b) have a length greater than that of other microstructures; and,
c) enter the dermis.

39. A method according to any one of the claims 1 to 38, wherein the microstructures include a material including at least one of:
a) a bioactive material;
b) a reagent for reacting with analytes in the subject;
c) a binding agent for binding with analytes of interest;
d) a probe for selectively targeting analytes of interest;
e) a material to reduce biofouling;
f) a material to attract at least one substance to the microstructures;
g) a material to repel at least one substance from the microstructures;
h) a material to attract at least some analytes to the projections; and,
i) a material to repel at least some analytes from the projections.

40. A method according to any one of the claims 1 to 39, wherein at least some of the microstructures are coated with a coating that at least one of:
a) modifies surface properties to at least one of:
i) increase hydrophilicity;
ii) increase hydrophobicity; and,
iii) minimize biofouling;
b) attracts at least one substance to the microstructures;
c) repels at least one substance from the microstructures;
d) acts as a barrier to preclude at least one substance from the microstructures; and,
e) includes at least one of:
i) polyethylene;
ii) polyethylene glycol;
iii) polyethylene oxide;
iv) zwitterions;
v) peptides;
vi) hydrogels; and,
vii) SAMs.
